(19) United States
(12) Patent Application Publication
(10) Pub. No.: US 2021/0145706 A1
(43) Pub. Date: May 20, 2021

(54) COSMETIC

(71) Applicant: Geol Chemical Co., Ltd., Nara (JP)

(72) Inventor: Keiji MORIMURA, Katsuragi-shi, Nara (JP)

(21) Appl. No.: 16/626,565

(22) PCT Filed: Aug. 2, 2018
(86) PCT No.: PCT/JP2018/029064

§ 371 Date: Dec. 25, 2019

(30) Foreign Application Priority Data
Aug. 2, 2017 (JP) 2017-150081

(51) Int. Cl. A61K 8/02; A61K 8/81; A61K 8/34; A61K 8/368; A61K 8/19; A61K 8/37; A61K 8/891; A61K 8/67; A61K 8/92; A61K 8/60; A61K 8/44; A61Q 19/00

(52) U.S. Cl. CPC A61K 8/0233; A61Q 19/007; A61K 8/345; A61K 8/368; A61K 8/19; A61K 8/375; A61K 8/37; A61K 8/891; A61K 8/342; A61K 8/678; A61K 8/922; A61K 8/60; A61K 8/34; A61K 8/44; A61K 8/8147

(57) ABSTRACT

Provided is a cosmetic product with superb aesthetics having functionalities of a plurality of cosmetic materials with different forms in a single cosmetic product.

Cosmetic product 1 of the present invention is completed by first loading a container 2 with a gel-like, cream-like or semisolid formulation A, and then loading a gel-like, cream-like or semisolid formulation B with a different composition inside the loaded formulation A. The present invention can provide cosmetic product 1 having a shape wherein the formulation B with a different functionality is surrounded in a spherical shape inside the formulation A having another functionality, the cosmetic product 1 having both superb aesthetics and functionalities.

### TECHNICAL FIELD

The present invention relates to a cosmetic product in which a transparent gel-like or semisolid formulation is loaded into a container wherein, inside of the formulation, a cream-like or semisolid formulation having a different composition is stably included in a spherical shape or a spheroid shape.

### BACKGROUND ART

Skin care products with various functionalities such as cream, milky lotion, lotion, beauty lotion, beauty gel, beauty oil, cleansing gel, cleansing cream, cleansing milky lotion, cleansing oil, masks, and cleansing foam are sold.

Basic components of these skin care products can be classified into an oily component highly compatible with oil, an aqueous component highly compatible with water, and further a moisturizing agent exhibiting a property between the aqueous component and the oily component.

An aqueous component contained in a cosmetic material replenishes moisture to the skin and has a role as a base which dissolves an aqueous active ingredient or cleansing component. Further, an oily component replenishes oil to the skin and has a role as a base which dissolves an oily active ingredient or cleansing component. Furthermore, a moisturizing component plays a role of keeping the moisture of the skin by retaining moisture or oil in the skin for a long time.

In general, aqueous lotion, beauty lotion, beauty gel and cleansing gel do not contain an oily component, and are used for the purpose of replenishing moisture or a moisturizing component to the skin, or mainly for the purpose of washing away an aqueous component of the skin. Beauty oil and cleansing oil do not contain an aqueous component, and are used for the purpose of replenishing oil to the skin or for the purpose of washing away an oily component of the skin.

However, these oily cosmetic materials have a greasy feeling and stickiness when in use. Emulsions emulsified by a surfactant have been used as cream, milky lotion, or cleansing cream for the purpose of replenishing an oily component and an aqueous component while suppressing the greasy feeling, or for the purpose of adding a component such as cleansing component which is both water soluble and oil soluble.

In this manner, shapes of skin care products are subdivided, and various types of skin care products are commercially sold. While each of the subdivided skin care products has a specialized function and each formulation has an excellent functionality, a user needs to use various types of skin care cosmetic materials in order depending on the situation, which often imposes a temporal or economic burden on the user.

Under this circumstance, cosmetic products having functionalities of multiple skin care products are also commercially sold. For example, a product which has a functionality of cream containing an oily component, a functionality of beauty lotion to which a moisturizing component or an active ingredient is added in abundance, and further a functionality of lotion containing a water soluble component, or a product which has a function of cream and a function of a mask confining components in the skin are sold.

However, functions are insufficiently exerted in any of these products, such as having an inferior effect of replenishing oil, or having an inferior moisturizing function, having an inferior function of a mask, or having an inferior moisturizing function. Many consumers cannot be satisfied with these products, and often use multiple products specialized in each function such as cream and beauty lotion, lotion or masks. Further, since a part of oil in a formulation of these products is emulsified in the formulation, a surfactant is added to the entire formulation, and more surfactant than necessary is added.

The surfactant described above may be a burden on the skin and may cause irritation. Further, some active ingredients such as salts are components that separate an emulsified formulation. It is difficult to combine these components. Furthermore, emulsification becomes difficult when the amount of an added moisturizing agent having a property between an oily component and an aqueous component is increased. Components that can be added to such a complex type of skin care product are greatly limited in formulation design. In this manner, it is technically difficult to configure one type of skin care product to sufficiently have multiple functionalities. In addition, due to the nature of the product, the outward appearance, i.e., aesthetics, of cosmetic products also leads to significant motivation for purchase of cosmetic products. Cosmetic products are often commercially sold with a formulation loaded in a container with superb aesthetics that incorporates various designs.

The amount of moisture and the amount of oil in the skin greatly change depending on the lifecycle of a person. Thus, the skin may need more aqueous components, or may need more oily components depending on the condition of the skin upon use. It is generally possible to deal with the change in the skin condition by using a cosmetic product to which more aqueous components such as lotion are added and a cosmetic product to which more oily components such as cream are added while adjusting the amount of each formulation used depending on the skin condition upon use.

Conventional cosmetic products having functions of multiple skin care products did not include any cosmetic product that can be used differently according to the skin condition in this manner. For example, Japanese Laid-Open Publication No. 2006-282588 (Patent Literature 1) proposes a bilayer type skin whitening cosmetic material which is separated into a transparent upper layer and a gel-like lower layer, when in still standing, has excellent aesthetics of the outward appearance, and has a good re-dispersibility and storage stability. However, it is not possible to use this cosmetic material while adjusting the amount of a cosmetic product to which more aqueous components are added and the amount of a cosmetic product used to which more oily components are added depending on the skin condition upon use.

Further, the cosmetic material described in Patent Literature 1 is separated into a water layer with fluidity in the upper layer portion and a gel layer in the lower layer, when in still standing. Said cosmetic material must be re-dispersed upon use. Thus, the feeling and functionality upon use are not greatly different from those of conventional cosmetic products. Further, the water layer part with high fluidity is merely located on top of the gel layer in the cosmetic material described in Patent Literature 1, when in a still standing state. It is difficult to consider that said cosmetic material is a product with superb aesthetics, when in a still standing state, in which the both components have no movement.

Japanese National Phase PCT Laid-Open Publication No. 2006-525229 (Patent Literature 2) proposes a striped personal cleansing composition wherein a cleansing phase and a benefit phase are packaged in physical contact with each other and maintain stability.

However, since the personal cleansing composition described in the above literature is striped, said composition cannot be used while adjusting the amount of a cosmetic product used to which more aqueous components are added and the amount of a cosmetic product used to which more oily components are added depending on the skin condition upon use.

Further, the composition of Patent Literature 2 is merely a cleansing composition in which a cleansing phase and a benefit phase comprising a high internal phase emulsion are simply loaded in a striped-shape. In a cosmetic product comprising this composition, no superb aesthetics can be found in the surface of the side, which constitutes the majority of the outer appearance of the product, other than the top. Furthermore, manufacturing a cosmetic product comprising the composition of Patent Literature 2 needs to simultaneously load a container such as a tube with two types of formulations by using a pump and a hose. Loading requires fine control, and working steps will be complicated.

Thus, there has been a demand for a cosmetic product that sufficiently has two different types of functionalities in a single skin care product as in a skin care product having a sufficient functionality of aqueous components, which is a nature of lotion or beauty lotion, and a sufficient functionality of oily components, which is a nature of cream. The subject matter disclosed in Japanese Patent No. 5670031 (Patent Literature 3) is excellent subject matter solving these problems. However, the composition of outer layer gel and inner layer cream which is used was not necessarily optimal.

### CITATION LIST

### Patent Literature

[PTL 1] Japanese Laid-Open Publication No. 2006-282588
[PTL 2] Japanese National Phase PCT Laid-Open Publication No. 2006-525229
[PTL 3] Japanese Patent No. 5670031

### SUMMARY OF INVENTION

### Technical Problem

The problem to be solved by the present invention is to optimize the composition of outer layer gel and inner layer cream used in Patent Literature 3 to provide a stable cosmetic product loaded with two formulations with superb aesthetics. The present invention provides an optimized composition of outer layer gel and inner layer cream. A cosmetic product loaded with two formulations having superb aesthetics and comprising an outer layer and an inner layer having superb stability is thereby provided.

The cosmetic product of the present invention is completed by first loading a container with a first gel-like, cream-like or semisolid formulation, and then loading a second gel, cream-like or semisolid formulation with a different composition inside the loaded formulation. The cosmetic product of the present invention prepared in this manner is a new type of skin care product in which inside of one formulation includes the other formulation in a spherical shape or a spheroid shape, wherein the optimized composition of outer layer gel and inner layer cream is one of the features.

Thus, the present invention provides the following.

### (Item 1)

A cosmetic product having a container, a transparent or semitransparent formulation A loaded into the container, and a formulation B embedded in the formulation A, wherein:

the formulation A is an aqueous gel, and the formulation B is an emulsified composition;

the shape of the formulation B is a shape selected from the group consisting of a perfectly spherical shape, a spherical shape, an egg shape, a spheroid shape, a shape having an oval cross-section, a perfectly spherical shape with a protrusion on a surface, a spherical shape with a protrusion on a surface, an egg shape with a protrusion on a surface, a spheroid shape with a protrusion on a surface, and a shape having an oval cross-section with a protrusion on a surface;

the formulation B is added at 10 to 60% by weight of an entire formulation amount;

the formulation A comprises acrylic acid/alkylmethacrylic acid copolymer and a moisturizing agent; and

the formulation B comprises
an oily component selected from the group consisting of glyceryl tri-2-ethylhexanoate, squalane, and olive oil,
cetyl 2-ethylhexanoate,
methyl polysiloxane,
behenyl alcohol,
cetanol,
lipophilic glyceryl monostearate,
hydrogenated soybean phospholipid,
acrylic acid/alkylmethacrylic acid copolymer,
moisturizing agent, and
N-stearoyl-L-sodium glutamate.

### (Item 2)

The cosmetic product of item 1, wherein the formulation A further comprises

a pH adjusting agent selected from the group consisting of potassium hydroxide, L-arginine, and triethanolamine, and

wherein the moisturizing agent is selected from the group consisting of concentrated glycerin, sorbit, dipropylene glycol, and 1,3-butylene glycol.

### (Item 3)

The cosmetic product of item 1, wherein the formulation A further comprises:
a pH adjusting agent selected from the group consisting of potassium hydroxide, L-arginine, and, triethanolamine; and
a preservative selected from the group consisting of 1,2-pentanediol, methyl parahydroxybenzoate, and phenoxyethanol, and

wherein the moisturizing agent is selected from the group consisting of concentrated glycerin, sorbit, dipropylene glycol, and 1,3-butylene glycol.

### (Item 4)

The cosmetic product of item 1, wherein the oily component is glyceryl tri-2-ethylhexanoate.

### (Item 5)

The cosmetic product of item 1, wherein the formulation B further comprises

a pH adjusting agent selected from the group consisting of potassium hydroxide, L-arginine, and triethanolamine, and

wherein the moisturizing agent is selected from the group consisting of concentrated glycerin, sorbit, dipropylene glycol, and 1,3-butylene glycol.

### (Item 6)

The cosmetic product of item 1, wherein the formulation B further comprises:
a pH adjusting agent selected from the group consisting of potassium hydroxide, L-arginine, and triethanolamine; and
a preservative selected from the group consisting of 1,2-pentanediol, methyl parahydroxybenzoate, and phenoxyethanol, and

wherein the moisturizing agent is selected from the group consisting of concentrated glycerin, sorbit, dipropylene glycol, and 1,3-butylene glycol.

### (Item 7)

The cosmetic product of item 5 or 6, wherein the formulation B further comprises an antioxidant selected from the group consisting of natural vitamin E and dibutylhydroxytoluene.

### (Item 8)

The cosmetic product of item 7, wherein the formulation B further comprises an emulsifying agent selected from the group consisting of maltitol laurate and maltitol hydroxyalkyl(12,14) ether.

### (Item 9)

The cosmetic product of item 7, wherein the formulation B further comprises xanthan gum.

### (Item 10)

The cosmetic product of item 8 or 9, wherein the formulation B further comprises dehydrated ethanol.

### (Item 11)

The cosmetic product of item 1, wherein the formulation B is embedded while floating in the formulation A.

### (Item 12)

The cosmetic product of item 1, wherein the formulation B is added at 20 to 40% by weight of an entire formulation amount.

### (Item 13)

The cosmetic product of item 1, wherein a maximum load indicating a formulation viscosity of the formulation A and the formulation B measured under the following conditions is 0.3 N to 2.0 N:

a maximum value of load applied per cross-sectional area of a rod-like probe with a diameter of 20 mm when the rod-like probe is inserted 25 mm at a rate of 60 mm/min as measured with Sun Scientific Co., Ltd.'s SUN RHEO METER (COMPAC 100-II).

The present invention also provides the following.

### (Item A1)

A cosmetic product having a container, a transparent or semitransparent formulation A loaded into the container, and a formulation B embedded in the formulation A, wherein:

the formulation A is an aqueous gel, and the formulation B is an emulsified composition;

the shape of the formulation B is a shape selected from the group consisting of a perfectly spherical shape, a spherical shape, an egg shape, a spheroid shape, a shape having an oval cross-section, a perfectly spherical shape with a protrusion on a surface, a spherical shape with a protrusion on a surface, an egg shape with a protrusion on a surface, a spheroid shape with a protrusion on a surface, and a shape having an oval cross-section with a protrusion on a surface;

the formulation B is added at 10 to 60% by weight of an entire formulation amount;

the formulation A comprises acrylic acid/alkylmethacrylic acid copolymer and a moisturizing agent, and the formulation B comprises glyceryl tri-2-ethylhexanoate, cetyl 2-ethylhexanoate, methyl polysiloxane, behenyl alcohol, cetanol, lipophilic glyceryl monostearate, hydrogenated soybean phospholipid, acrylic acid/alkylmethacrylic acid copolymer, moisturizing agent, and N-stearoyl-L-sodium glutamate.

### (Item A2)

The cosmetic product of item A1, wherein the formulation A comprises

acrylic acid/alkylmethacrylic acid copolymer,

potassium hydroxide,

concentrated glycerin, and

1,3-butylene glycol.

### (Item A3)

The cosmetic product of item A1, wherein the formulation A comprises

acrylic acid/alkylmethacrylic acid copolymer,

potassium hydroxide,

concentrated glycerin,

1,3-butylene glycol, and

methyl parahydroxybenzoate.

### (Item A4)

The cosmetic product of item A1, wherein the formulation A comprises

acrylic acid/alkylmethacrylic acid copolymer,

potassium hydroxide,

concentrated glycerin,

1,3-butylene glycol,

methyl parahydroxybenzoate, and

phenoxyethanol.

### (Item A5)

The cosmetic product of item A1, wherein the formulation A comprises

acrylic acid/alkylmethacrylic acid copolymer,

potassium hydroxide,

concentrated glycerin,

1,3-butylene glycol, and

1,2-pentanediol.

### (Item A6)

The cosmetic product of item A1, wherein the formulation B comprises

glyceryl tri-2-ethylhexanoate,

cetyl 2-ethylhexanoate,

methyl polysiloxane,

behenyl alcohol,

cetanol,

lipophilic glyceryl monostearate,

natural vitamin E,

hydrogenated soybean phospholipid,

acrylic acid/alkylmethacrylic acid copolymer,

potassium hydroxide,

concentrated glycerin,

1,3-butylene glycol, and

N-stearoyl-L-sodium glutamate.

### (Item A7)

The cosmetic product of item A1, wherein the formulation B comprises

glyceryl tri-2-ethylhexanoate,

cetyl 2-ethylhexanoate,

methyl polysiloxane,

behenyl alcohol,

cetanol,

lipophilic glyceryl monostearate,

propyl parahydroxybenzoate,

natural vitamin E,

hydrogenated soybean phospholipid,

acrylic acid/alkylmethacrylic acid copolymer,

potassium hydroxide,

concentrated glycerin,

1,3-butylene glycol,

maltitol laurate,

dehydrated ethanol,

N-stearoyl-L-sodium glutamate, and

ester parahydroxybenzoate.

### (Item A8)

The cosmetic product of item A1, wherein the formulation B comprises

glyceryl tri-2-ethylhexanoate,

cetyl 2-ethylhexanoate,

methyl polysiloxane,

behenyl alcohol,

cetanol,

lipophilic glyceryl monostearate,

propyl parahydroxybenzoate,

natural vitamin E,

hydrogenated soybean phospholipid,

acrylic acid/alkylmethacrylic acid copolymer,

xanthan gum,

potassium hydroxide,

concentrated glycerin,

1,3-butylene glycol,

phenoxyethanol,

N-stearoyl-L-sodium glutamate, and

propyl parahydroxybenzoate.

### (Item A9)

The cosmetic product of item A1, wherein the formulation B comprises

glyceryl tri-2-ethylhexanoate,

cetyl 2-ethylhexanoate,

methyl polysiloxane,

behenyl alcohol,

cetanol,

lipophilic glyceryl monostearate,

1,2-pentanediol,

natural vitamin E,

hydrogenated soybean phospholipid,

acrylic acid/alkylmethacrylic acid copolymer,

xanthan gum,

potassium hydroxide,

concentrated glycerin,

1,3-butylene glycol,

maltitol laurate,

dehydrated ethanol, and

N-stearoyl-L-sodium glutamate.

### (Item A10)

The cosmetic product of item A1, wherein the formulation B is embedded while floating in the formulation A.

### (Item A11)

The cosmetic product of item A1, wherein the formulation B is added at 20 to 40% by weight of an entire formulation amount.

### (Item A12)

The cosmetic product of item A1, wherein a maximum load indicating a formulation viscosity of the formulation A and the formulation B measured under the following conditions is 0.3 N to 2.0 N:

a maximum value of load applied per cross-sectional area of a rod-like probe with a diameter of 20 mm when the rod-like probe is inserted 25 mm at a rate of 60 mm/min as measured with Sun Scientific Co., Ltd.'s SUN RHEO METER (COMPAC 100-II).

For example, the concentration of each component listed in the Examples may vary in the range of ±101, ±8%, ±6%, ±4%, ±2%, 1%, or ±0.5% relative to a specific numerical value of each component concentration described in the Examples. Further, not all components used in the Examples necessarily need to be included in manufacturing the cosmetic product of the present invention. Those skilled in the art do not need to use a part of the components listed in the Examples in manufacturing a cosmetic product loaded with two formulations comprising an outer layer and an inner layer, which is a feature of the present invention, as required.

### Advantageous Effects of Invention

In the cosmetic product of the present invention, a transparent or semitransparent formulation A is loaded into a transparent or semitransparent container, a formulation B is embedded in formulation A, formulations A and B are each gel-like, cream-like or semisolid, formulations A and B have different compositions, and at least a part of the surface of formulation B has a protruding curved surface. Having such a shape can provide a cosmetic with the following advantages. The advantages of the present inventions are listed below.

(1) The present invention can provide a cosmetic product with functionalities of two types of skin care products selected from skin care cosmetic materials such as cream, aqueous gel, beauty gel, oily gel, aqueous cleansing gel, oily cleansing gel, cleansing cream, mask formulation, or cleansing foam in one type of formulation.
(2) In the cosmetic product of the present invention, formulation A and formulation B with functionalities of two different types of cosmetic materials are combined while being separated. Since a component contained in one of the formulations does not have a significant effect on the other formulation, selectivity of components that can be added to each formulation is improved.
(3) When one of the formulations includes an emulsified composition, a formulation which is not an emulsified composition is separated from a formulation of an emulsified composition. Thus, it is sufficient to add a surfactant used for emulsification of oily components of the emulsified composition only in the amount necessary for emulsifying the oil inside. The amount of a surfactant used in a cosmetic material in which the formulations are combined can be decreased.
(4) When the emulsified composition is included inside or in the outer layer part (e.g., inside), a component which is originally difficult to be added to an emulsified composition as in a component which separates an emulsified composition as in salts or a moisturizing agent having a property between an aqueous component and an oily component can be added to the other formulation.
(5) In the present invented product, two types of formulations with visually different shapes are commonly combined while being separated in a transparent or semitransparent container, wherein the inner formulation has a protruding curved surface at least on the surface thereof. A user also can use the product by visually adjusting the amount of two types of formulations used depending on the skin condition upon use. Specifically, when a formulation of an emulsified composition is embedded in highly transparent aqueous gel, a user can use the product while making visual adjustments, such as using more of the aqueous gel part in the perimeter portion or more of the emulsified composition containing more oil depending on the skin condition. Despite being a single product, the product can be used differently according to the skin condition.
(6) In the cosmetic product of the present invention, a formulation in the inner layer has a shape that is completely enveloped by a formulation in the outer layer. Such a shape can dramatically reduce outflow of fragment components such as fragrance or essential oil to the outside of the formulation up to immediately prior to use by adding the fragment components only to the formulation in the inner layer part. A cosmetic product of the present invention, which spreads the fresh scent as of immediately after manufacturing upon use of the cosmetic product, can be provided. Furthermore, the probability of being in directly contact with air is extremely low for a formulation in a shape that includes an oily component inside, so that oxidation of the oily component inside also can be suppressed.
(7) A cosmetic product using a highly transparent aqueous gel as the formulation in the outer layer portion and using a colored gel or a colored emulsified composition as the formulation in the inner layer portion has better aesthetics as well. It is possible to provide a formulation with superb aesthetics and a mystical form in which a spherical emulsified layer or gel layer is completely surrounded by gel, depending on the shape of the container, ratio of the aqueous gel in the outer layer portion and the formulation inside that are added, and presence/absence of color.

As described above, the present invented product can provide a cosmetic product with various functionalities that existing products do not have.

### BRIEF DESCRIPTION OF DRAWINGS

FIG. 1 (a) to (d) of FIG. 1 are schematic diagrams depicting various embodiment of the cosmetic product of the present invention.

FIG. 2A to FIG. 2C show photographs of Comparative Example 1 upon manufacturing.

FIG. 2A to FIG. 2C show photographs of Comparative Example 1 upon manufacturing.

FIG. 2A to FIG. 2C show photographs of Comparative Example 1 upon manufacturing.

FIG. 3A to FIG. 3C show photographs of Comparative Example 1 after 90 days from manufacturing.

FIG. 3A to FIG. 3C show photographs of Comparative Example 1 after 90 days from manufacturing.

FIG. 3A to FIG. 3C show photographs of Comparative Example 1 after 90 days from manufacturing.

FIG. 4 shows a photograph of Example 1 upon manufacturing.

FIG. 5 shows a photograph of Example 1 after 90 days from manufacturing.

FIG. 6 shows a photograph of Example 2 upon manufacturing.

FIG. 7 FIG. 7 shows a photograph of Example 2 after 90 days from manufacturing.

FIG. 8 shows a photograph of Example 3 upon manufacturing.

FIG. 9 shows a photograph of Example 3 after 90 days from manufacturing.

FIG. 10 shows a photograph of Example 4 upon manufacturing.

FIG. 11 shows a photograph of Example 4 after 90 days from manufacturing.

FIG. 12 shows a photograph of Example 5 upon manufacturing.

FIG. 13 shows a photograph of Example 5 after 90 days from manufacturing.

FIG. 14 shows a photograph of Example 6 upon manufacturing.

FIG. 15 shows a photograph of Example 6 after 90 days from manufacturing.

FIG. 16 shows a photograph of Example 7 upon manufacturing.

FIG. 17 shows a photograph of Example 7 after 90 days from manufacturing.

FIG. 18 shows a photograph of Example 8 upon manufacturing.

FIG. 19 shows a photograph of Example 8 after 90 days from manufacturing.

FIG. 20 shows a photograph of Example 9 upon manufacturing.

FIG. 21 shows a photograph of Example 9 after 90 days from manufacturing.

FIG. 22 shows a photograph of Example 10 upon manufacturing.

FIG. 23 shows a photograph of Example 10 after 90 days from manufacturing.

FIG. 24 shows a photograph of Example 11 upon manufacturing.

FIG. 25 shows a photograph of Example 11 after 90 days from manufacturing.

FIG. 26 shows a photograph of Example 12 upon manufacturing.

FIG. 27 shows a photograph of Example 12 after 90 days from manufacturing.

FIG. 28 shows a photograph of Example 13 upon manufacturing.

FIG. 29 shows a photograph of Example 13 after 90 days from manufacturing.

FIG. 30 shows a photograph of Example 14 upon manufacturing.

FIG. 31 shows a photograph of Example 14 after 90 days from manufacturing.

FIG. 32 shows a photograph of Example 15 upon manufacturing.

FIG. 33 shows a photograph of Example 15 after 90 days from manufacturing.

FIG. 34 shows a photograph of Example 16 upon manufacturing.

FIG. 35 shows a photograph of Example 16 after 90 days from manufacturing.

FIG. 36 shows a photograph of Example 17 upon manufacturing.

FIG. 37 shows a photograph of Example 17 after 90 days from manufacturing.

FIG. 38 shows a photograph of Example 18 upon manufacturing.

FIG. 39 shows a photograph of Example 18 after 90 days from manufacturing.

FIG. 40 shows a photograph of Example 19 upon manufacturing.

FIG. 41 shows a photograph of Example 19 after 90 days from manufacturing.

FIG. 42 shows a photograph of Example 20 upon manufacturing.

FIG. 43 shows a photograph of Example 20 after 90 days from manufacturing.

### DESCRIPTION OF EMBODIMENTS

The present invention is explained hereinafter. The terms used herein should be used in the meaning that is commonly used in the art, unless specifically noted otherwise. Thus, unless defined otherwise, all terminologies and scientific technical terms that are used herein have the same meaning as the general understanding of those skilled in the art to which the present invention pertains. In case of a contradiction, the present specification (including the definitions) takes precedence. Further, unless specifically and explicitly noted, concentration (%) means weight/weight percent.

### DEFINITION OF TERMS

The term “spherical” as used herein is not limited to a perfectly spherical shape, but also includes slightly deformed spherical shape (e.g., egg shape), spheroid shape (e.g., shape having an oval cross-section), spherical shape with a small protrusion on the surface, and the like.

Similarly, “semispherical shape” as used herein is not limited to a perfectly semispherical shape, but also includes a slightly deformed semispherical shape, a semispherical shape with a small protrusion on the surface, and the like.

The term “emulsion” as used herein refers to a homogenous mixture of a liquid, semisolid, or solid which do not dissolve into each other as in an aqueous component and an oily component.

The term “emulsified composition” as used herein refers to a homogenous mixture of liquid, semisolids, or solids which do not dissolve into each other as in an aqueous component and an oily component.

As used herein, the term “aqueous component” refers to a component that can be dissolved and/or diluted by water.

As used herein, the term “oily component” refers to a substance, which is a liquid or solid at normal temperature, is insoluble to water, is viscous, has a lower specific gravity than water, and is flammable.

Any water soluble substance or any substance or solid that can be dispersed in gel can be dispersed in the formulation in the outer layer portion and the inner formulation of the present invention. Examples of components that can be added to a formulation can include cosmetic product substrates such as fats and oils, waxes, hydrocarbons, silicones, fatty acids, alcohols, esters, surfactants, thickeners, and powders, as well as pharmaceuticals, active ingredients of quasi drugs, pH adjusting agents, preservatives, pigments, fragrance, antioxidants, and naturally derived extracts.

The color tone of two types of formulations constituting the present invention does not matter. The formulations may be opaque, white, transparent, or colored gel or emulsified compositions. From the viewpoint of aesthetics, the formulation in the perimeter portion is desirably transparent or semitransparent with color or no color, especially when the formulation is an aqueous gel.

For example, the “oily component” used in the present invention includes, but is not limited to, a substance selected from the group consisting of the following: fats and oils such as avocado oil, almond oil, olive oil, camellia oil, sesame oil, rice bran oil, safflower oil, soybean oil, corn oil, rapeseed oil, apricot kernel oil, persic oil, peach kernel oil, castor oil, sunflower oil, grape seed oil, cottonseed oil, coconut oil, wheat germ oil, rice germ oil, evening primrose oil, hybrid sunflower oil, macadamia nut oil, meadowfoam oil, hazelnut oil, palm kernel oil, palm oil, coconut oil, cocoa butter, shea butter, wood wax, mink oil, turtle oil, egg yolk oil, beef tallow, milk fat, lard, and horse oil; waxes such as jojoba oil, carnauba wax, candelilla wax, rice bran wax, orange roughy oil, beeswax, shellac, lanolin, and montan wax; hydrocarbons such as squalene, squalane, liquid paraffin, paraffin, microcrystalline wax, Vaseline, soft fluid isoparaffin, hydrogenated polyisobutylene, ozocerite, ceresin, α-olefin oligomer, polybutene, and polyethylene; higher fatty acids such as lauric acid, myristic acid, palmitic acid, stearic acid, behenic acid, hydroxystearic acid, oleic acid, linoleic acid, ethylhexanoic acid, isostearic acid, isopalmitic acid, isotridecanoic acid, isononanoic acid, and pentadecanoic acid; higher alcohols such as lauryl alcohol, stearyl alcohol, cetearyl alcohol, behenyl alcohol, cetanol, oleyl alcohol, lanolin alcohol, cholesterol, isocholesterol, sitosterol, stigmasterol, isostearyl alcohol, octyldodecanol, and hexyldecanol; esters such as isopropyl myristate, isopropyl palmitate, butyl stearate, ethyl oleate, cetyl palmitate, myristyl myristate, octyldodecyl myristate, octyldodecyl oleate, cholesteryl stearate, cholesteryl hydroxystearate, tricaprin, trimyristin, glyceryl tri-2-ethylhexanoate (trioctanoin), isopropyl isostearate, isopropyl isostearate, ethyl isostearate, cetyl ethylhexanoate, stearyl ethylhexanoate, glyceryl triethylhexanoate, glyceryl triethylhexanoate, trimethylolpropane triethylhexanoate, pentaerythril tetraethylhexanoate, glyceryl triisostearate, trimethylolpropane triisostearate, pentaerythril tetraisostearate, pentaerythril triisostearate, isocetyl isostearate, octyldodecyl dimethyl octanoate, myristyl lactate, cetyl lactate, trioctyldodecyl citrate, and diisostearyl malate; and a mixture of two of more thereof.

A “moisturizing component” may be added to the cosmetic material of the present invention. For example, such a “moisturizing component” includes, but is not limited to, a substance selected from the group consisting of the following. As used herein, the term “moisturizing component” is interchangeably used with “moisturizing agent”.

Glycerin (e.g., concentrated glycerin such as glycerin aqueous solution of 85% by weight or more), 1,3-butylene glycol, sorbit, propylene glycol, 3-methyl-1,3-butanediol, 1,3-propanediol, 2-methyl-1,3-propanediol, trimethylolpropane, pentaerythritol, hexylene glycol, diglycerin, polyglycerin, diethylene glycol, polyethylene glycol, dipropylene glycol, polypropylene glycol, ethylene glycol, diethylene glycol monoethyl ether (ethoxydiglycol), ethylene glycol monoethyl ether, ethylene glycol monobutyl ether, diethylene glycol dibutyl ether, sorbitol, xylitol, erythritol, mannitol, maltitol, glucose, fructose, galactose, mannose, threose, xylose, arabinose, fucose, ribose, deoxyribose, maltose, trehalose, lactose, raffinose, gluconic acid, glucuronic acid, cyclodextrin, β-glucan, chitin, chitosan, heparin and derivatives thereof, pectin, arabinogalactan, dextrin, dextran, glycogen, ethyl glucoside, glucosyl ethyl methacrylate polymer, hyaluronic acid, sodium hyaluronate, mucoitin sulfuric acid, charonin sulfate, keratosulfate, dermatan sulfate, Tremella fuciformis extract, Tremella fuciformis polysaccharide, tuberose polysaccharide, citric acid, tartaric acid, urea, salts of 2-pyrrolidone-5-carboxylic acid and sodium thereof, betaine (trimethylglycine), proline, hydroxyproline, arginine, lysine, serine, glycine, alanine, phenylalanine, tyrosine, β-alanine, threonine, glutamic acid, glutamine, asparagine, aspartic acid, cysteine, cystine, methionine, leucine, isoleucine, valine, tryptophan, histidine, taurine, collagen, atelocollagen, gelatin, elastin, peptide from decomposed collagen, hydrolyzed collagen, hydroxypropylammonium chloride hydrolyzed collagen, peptide from decomposed elastin, peptide from decomposed keratin, hydrolyzed keratin, peptide from decomposed conchiolin, hydrolyzed conchiolin, peptide from decomposed silk protein, hydrolyzed silk, sodium lauroyl hydrolyzed silk, peptide from decomposed soybean protein, peptide from decomposed wheat protein, hydrolyzed wheat protein, peptide derived from decomposed casein, acylated peptide, other protein peptides and derivatives thereof; palmitoyl oligopeptide, palmitoyl pentapeptide, palmitoyl tetrapeptide, lactic acid bacteria culture, yeast extract, eggshell membrane protein, bovine submaxillary mucin, hypotaurine, sesame lignan glycoside, glutathione, albumin, and whey; choline chloride and phosphorylcholine; and placenta extract, elastin, collagen, aloe extract, Hammamelis virginiana water, Luffa cylindrica water, Chamomilla recutita extract, licorice extract, comfrey extract, silk extract, Rosa roxburghii extract, Achillea millefolium extract, Eucalyptus globulus extract, melilot extract, natural ceramides (types 1, 2, 3, 4, 5, and 6), hydroxyceramide, and glycosphingolipid.

A “pH adjusting agent” may be added to the cosmetic material of the present invention as required to adjust pH. For example, such a “pH adjusting agent” includes, but is not limited to, a substance selected from the group consisting of sodium hydroxide, potassium hydroxide, calcium hydroxide, magnesium hydroxide, sodium carbonate, potassium carbonate, calcium carbonate, ammonium carbonate, ammonia, morpholine, triethanolamine, diethanolamine, dimethylamine, diethylamine, trimethylamine, triethylamine, and arginine (e.g., L-arginine).

An “antioxidant” may be added to the cosmetic material of the present invention as required. For example, such an “antioxidant” includes, but is not limited to, a substance selected from the group consisting of phenols, hydroquinones, benzoquinones, aromatic amines, or vitamins. Alternatively, for example, an antioxidant includes, but is not limited to, BHT(2,6-di-t-butyl-p-cresol), vitamin C, and vitamin E. Alternatively, an antioxidant includes, but is not limited to, butylhydroxyanisole, butylhydroxytoluene, propyl gallate, natural vitamin E and derivatives thereof, Oryza sativa extract, and white mustard hydrolyzed extract.

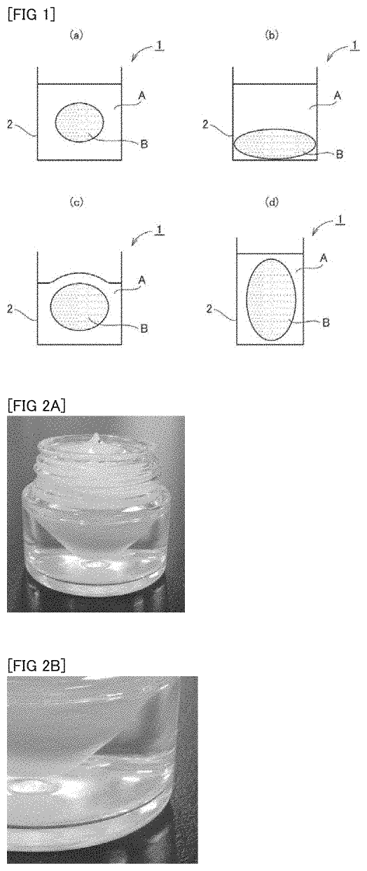

As shown in FIG. 1, a cosmetic product 1 of the present invention has a container 2 (which is preferably transparent or semitransparent, but does not necessarily need to be transparent or semitransparent), a transparent or semitransparent formulation A loaded into the container 2, and a formulation B embedded in formulation A.

Formulations A and B are each gel-like, cream-like, or semisolid. Formulations A and B have different compositions, and at least a part of the surface of formulation B has a protruding curved surface. Preferably, formulation A is transparent gel-like, and formulation B is cream-like.

Formulation B may be spherical. In such a case, formulation B can be embedded while floating in formulation A as shown in FIG. 1(a).

As shown in FIG. 1(b), the bottom portion of formulation B may be in contact with the bottom surface of the container 2. In such a case, the top portion of formulation B would be nearly semispherical. In this regard, formulation B contacts the bottom surface of the container 2 directly or through a thin layer of formulation A.

Further, as shown in FIG. 1(c), the central portion of the cosmetic material may be bulging out while formulation B is surrounded by formulation A.

The shape of the container 2 and the amount of formulations loaded can be optionally changed. For example, as shown in FIG. 1(d), formulation B may have a long oval shape in the longitudinal direction.

A cosmetic product can be commonly manufactured by inserting a nozzle inside pre-loaded formulation A and loading formulation B. Depending on the combination of formulation A and formulation B, a protrusion may be formed on top of the surface forming a protruding curved surface of formulation B when withdrawing the nozzle after loading formulation B, but this would not impair the aesthetics of the product or have any effect on the completion of the invention.

The present invention can provide a novel skin care product having the functionalities of two types of formulations, formulation A and formulation B, in a single product by loading formulations with two types of functionalities in two phases. Further, the present invention can provide a novel skin care product, which uses formulation A such as aqueous gel with high transparency in the perimeter portion so that formulation B having a curved surface can be visually recognized from the outside through the container 2 and formulation A, thus having superb aesthetics.

A preferred combination of two types of formulations is “aqueous gel” in the outer layer and “cream” inside, or “beauty gel” in the outer layer and “cream” inside.

While the viscosity of each formulation is not particularly limited, if the viscosity is too low, an inner emulsified composition or gel moves to or mixes with an aqueous gel part in the perimeter portion due to passage of time or an impact or the like, such that the shape cannot be stably maintained. If the viscosity is too high, it is difficult to load a formulation and such high viscosity impairs the usability of a product.

In any case, a formulation that does not have fluidity and is not too hard is desirable. Furthermore, a formulation in the outer layer portion and an inner formulation are desirably, but are not limited to, formulations with similar levels of viscosity in view of the ease of use of the formulations, ease of loading in the production process, and need for loading the inner formulation in an approximately spherical shape.

### (Preferred Composition of an Outer Layer)

For example, a preferred composition of an outer layer used in the present invention is, but not limited to, the following. For example, the concentration of each component listed below may vary in the range of ±10%, ±8%, ±6%, ±4%, ±2%, ±1% or ±0.5% with respect to the specific numerical value of each component concentration that is specifically described. Further, in manufacturing the cosmetic product of the present invention, the product does not necessarily need to contain all of the components listed below. A required component is acrylic acid/alkylmethacrylic acid copolymer. Potassium hydroxide can be replaced with an alkaline substance such as sodium hydroxide or arginine. Concentrated glycerin (e.g., glycerin aqueous solution of 85% by weight or more) and 1,3-butylene glycol can be replaced with polyhydric alcohols such as propylene glycol. Methyl parahydroxybenzoate of Prescription 2-1 and phenoxyethanol of Prescription 3-1 also can be replaced with a preservative such as ethyl parahydroxybenzoate, benzoic acid, sodium benzoate, sodium dehydroacetate or hinokitiol. 1,2-pentanediol of Prescription 4-1 also can be replaced with polyhydric alcohols having a preservative effect such as 1,2-hexanediol or 1,2-octanediol. Those skilled in the art appropriately do not need to use a part of the components listed below in manufacturing a cosmetic product loaded with two formulations comprising an outer layer and an inner layer, which is a feature of the present invention.

### (Outer Layer Gel Prescription: Prescription 2-1)

The optimized outer layer prescription of the present invention includes, for example, Prescription 2-1 shown below.

(A) Acrylic acid/alkylmethacrylic acid copolymer: 0.7 (i by weight)
(A) Concentrated glycerin: 15 (% by weight)
(A) 1,3-butylene glycol: 5 (% by weight)
(A) Methyl parahydroxybenzoate: 0.15 (% by weight)
(A) Purified water: added as the remainder so that the total amount would be 100 (% by weight)
(B) Potassium hydroxide: 0.26 (% by weight)

The outer layer prescription of the above composition is prepared by heating and dissolving component (A) while stirring, then adding component (B) dissolved to be 10% using purified water, and then cooling the mixture to room temperature while stirring, resulting in a transparent gel.

### (Outer Layer Gel Prescription: Prescription 3-1)

The optimized outer layer prescription of the present invention includes, for example, Prescription 3-1 shown below.

(A) Acrylic acid/alkylmethacrylic acid copolymer: 0.7 (i by weight)
(A) Concentrated glycerin: 15 (% by weight)
(A) 1,3-butylene glycol: 5 (% by weight)
(A) Methyl parahydroxybenzoate: 0.15 (% by weight) (A) Phenoxyethanol: 0.3 (% by weight)
(A) Purified water: added as the remainder so that the total amount would be 100 (% by weight)
(B) Potassium hydroxide: 0.26 (% by weight)

The outer layer prescription of the above composition is prepared by heating and dissolving component (A) while stirring, then adding component (B) dissolved to be 10% using purified water, and then cooling the mixture to room temperature while stirring, resulting in a transparent gel.

(Outer layer gel prescription: Prescription 4-1)

The optimized outer layer prescription of the present invention includes, for example, Prescription 4-1 shown below.

(A) Acrylic acid/alkylmethacrylic acid copolymer: 0.7 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)
(B) Potassium hydroxide: 0.26 (% by weight)

The outer layer prescription of the above composition is prepared by heating and dissolving component (A) while stirring, then adding component (B) dissolved to be 10% using purified water, and then cooling the mixture to room temperature while stirring, resulting in a transparent gel.

### (Preferred Composition of an Inner Layer)

For example, a preferred composition of an inner layer used in the present invention is, but not limited to, the following. For example, the concentration of each component listed below may vary in the range of ±10%, ±8%, ±6%, ±4%, ±2%, ±1% or ±0.5% with respect to the specific numerical value of each component concentration that is specifically described. Further, in manufacturing the cosmetic product of the present invention, the product does not necessarily need to contain all of the components listed below. Required components are lipophilic glyceryl monostearate, hydrogenated soybean phospholipid, acrylic acid/alkylmethacrylic acid copolymer and N-stearoyl-L-sodium glutamate. Glyceryl tri-2-ethylhexanoate, cetyl 2-ethylhexaonate and methyl polysiloxane also can be replaced with an oil component (oily component) including oils and fats such as avocado oil, almond oil, olive oil, camellia oil, sesame oil, rice bran oil, safflower oil, soybean oil, corn oil, rapeseed oil, apricot kernel oil, persic oil, peach kernel oil, castor oil, sunflower oil, grape seed oil, cottonseed oil, coconut oil, wheat germ oil, rice germ oil, evening primrose oil, hybrid sunflower oil, macadamia nut oil, meadowfoam oil, hazelnut oil, palm kernel oil, palm oil, coconut oil, cocoa butter, shea butter, wood wax, mink oil, turtle oil, egg yolk oil, beef tallow, milk fat, lard, and horse oil; waxes such as jojoba oil, carnauba wax, candelilla wax, rice bran wax, orange roughy oil, beeswax, shellac, lanolin, and montan wax; hydrocarbons such as squalene, squalane, liquid paraffin, paraffin, microcrystalline wax, Vaseline, soft fluid isoparaffin, hydrogenated polyisobutylene, ozocerite, ceresin, α-olefin oligomer, polybutene, and polyethylene. Behenyl alcohol and cetanol also can be replaced with higher alcohols such as lauryl alcohol, stearyl alcohol or cetearyl alcohol. Concentrated glycerin (e.g., glycerin aqueous solution of 85% by weight or more) and 1,3-butylene glycol can be replaced with polyhydric alcohols such as propylene glycol. Methyl parahydroxybenzoate of Prescription 2-2, and propyl parahydroxybenzoate and phenoxyethanol of Prescription 3-2 also can be replaced with a preservative such as ethyl parahydroxybenzoate, benzoic acid, sodium benzoate, sodium dehydroacetate or hinokitiol. 1,2-pentanediol of Prescription 4-2 also can be replaced with polyhydric alcohols having a preservative effect such as 1,2-hexanediol or 1,2-octanediol. Those skilled in the art appropriately do not need to use a part of the components listed below in manufacturing a cosmetic product loaded with two formulations comprising an outer layer and an inner layer, which is a feature of the present invention.

(Inner layer cream prescription: Prescription 2-2)

The optimized inner layer prescription of the present invention includes, for example, Prescription 2-2 shown below.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Propyl parahydroxybenzoate: 0.2 (% by weight)
(A) Natural vitamin E: 0.1 (% by weight)
(A) Hydrogenated soybean phospholipid: 0.3 (% by weight)
(B) Acrylic acid/alkylmethacrylic acid copolymer: 0.23 (% by weight)
(B) Concentrated glycerin: 10 (% by weight)
(B) 1,3-butylene glycol: 5 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Methyl parahydroxybenzoate: 0.2 (% by weight)
(B) Purified water: added as the remainder so that the total amount would be 100 (i by weight)
(C) Potassium hydroxide: 0.081 (% by weight)

The inner layer prescription of the above composition is prepared by separately heating component (A) and component (B) and mixing them at 80° C., adding component (C) dissolved to be 10% using purified water, stirring the mixture using a homomixer, and stirring the mixture while cooling it to room temperature, resulting in a white and cream-like emulsified composition.

### (Inner Layer Cream Prescription: Prescription 3-2)

The optimized inner layer prescription of the present invention includes, for example, Prescription 3-2 shown below.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Propyl parahydroxybenzoate: 0.2 (% by weight)
(A) Natural vitamin E: 0.1 (% by weight)
(A) Hydrogenated soybean phospholipid: 0.1 (% by weight)
(B) Acrylic acid/alkylmethacrylic acid copolymer: 0.23 (% by weight)
(B) Xanthan gum: 0.1 (% by weight)
(B) Concentrated glycerin: 10 (% by weight)
(B) 1,3-butylene glycol: 5 (% by weight)
(B) Phenoxyethanol: 0.3 (% by weight)
(B) N-stearoyl-L-sodium glutamate: 0.8 (% by weight)
(B) Methyl parahydroxybenzoate: 0.2 (% by weight)
(B) Purified water: added as the remainder so that the total amount would be 100 (% by weight)
(C) Potassium hydroxide: 0.081 (% by weight)
The inner layer prescription of the above composition is prepared by separately heating component (A) and component (B) and mixing them at 80° C., adding component (C) dissolved to be 10% using purified water, stirring the mixture using a homomixer, and stirring the mixture while cooling it to room temperature, resulting in a white and cream-like emulsified composition.

### (Inner Layer Cream Prescription: Prescription 4-2)

The optimized inner layer prescription of the present invention includes, for example, Prescription 4-2 shown below.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) Acrylic acid/alkylmethacrylic acid copolymer: 0.23 (% by weight)
(B) Xanthan gum: 0.1 (% by weight)
(B) Concentrated glycerin: 10 (% by weight)
(B) 1,3-butylene glycol: 1 (% by weight)
(B) 1,2-pentanediol: 4 (% by weight)
(B) Maltitol laurate: 0.34 (% by weight)
(B) Dehydrated ethanol: 0.07 (by weight)
(B) N-stearoyl-L-sodium glutamate: 0.5 (% by weight)
(B) Purified water: added as the remainder so that the total amount would be 100 (% by weight)
(C) Potassium hydroxide: 0.081 (% by weight)
The inner layer prescription of the above composition is prepared by separately heating component (A) and component (B) and mixing them at 80° C., adding component (C) dissolved to be 10% using purified water, stirring the mixture using a homomixer, and stirring the mixture while cooling it to room temperature, resulting in a white and cream-like emulsified composition.

### (Combination of an Outer Layer and an Inner Layer)

Preferred combinations of the outer layer and the inner layer shown above are as follows.
* Combination of the outer layer 2-1 and the inner layer 2-2,
* combination of the outer layer 3-1 and the inner layer 3-2, and
* combination of the outer layer 4-1 and the inner layer 4-2.

The viscosity of a formulation can be represented by the maximum load according to the following measurement method. The maximum load of a formulation is preferably 0.3 N to 2.0 N, and further preferably 0.5 N to 1.5 N. If the maximum load is less than 0.3 N, it is difficult to maintain a stable shape of a formulation as described above. If the maximum load is greater than 2.0 N, it is difficult to load a formulation or such a high viscosity impairs the usability of a product.

The maximum load of each formulation is measured with SUN RHEO METER (COMPAC 100-II) purchased from Sun Scientific Co., Ltd., which measures the maximum value of load applied per cross-sectional area of a rod-like probe with a diameter of 20 mm when the rod-like probe is inserted 25 mm at a rate of 60 mm/min.

While the specific gravity of each formulation varies depending on the application and prescription of each formulation, the specific gravity of formulation A is preferably 0.8 to 1.3, and further preferably 0.9 to 1.2. Further, the specific gravity of formulation B is preferably 0.7 to 1.2, and further preferably 0.8 to 1.1.

The cosmetic product of the present invention is completed by first loading a container with a formulation in the outer layer portion, and then loading the other formulation inside the formulation in the outer layer portion. In this regard, the method of loading is not limited. The cosmetic product of the present invention can be obtained by loading each formulation using a loading machine, an injector or the like that is commonly used in a manufacturing process of cosmetic products while being rotated or directly.

In the present invention, the ratio of the amount of the formulation first loaded into a container and the amount of the formulation loaded in the second phase is not particularly limited. The ratio should be determined depending on the functionality of the type of a selected formulation, or the combination of the cosmetic products of the present invention which is finally demanded.

From the viewpoint of the outward appearance of the present invention, the amount of inner formulation B which is loaded in the second phase is desirably 10% to 60% and more desirably 20 to 40% of the entire formulations. If the amount of formulation B is less than 10% of the entire formulations, the function of formulation B may not be exerted. If the amount exceeds 60%, the function of formulation A may not be exerted and the outward appearance may be impaired.

The formulation loaded in the second phase needs to be loaded inside the formulation that has been first loaded, but the location to where the formulation is loaded in the second phase is not particularly limited.

It is generally desirable to load the formulation near the center of the formulation that has been loaded first, but said formulation may be loaded at a position above or below the center of the formulation that has been loaded first depending on the hardness, shape, or functionality of the formulation, or the ratio at which the formulation is added. By loading said formulation above the center, said formulation is less likely to adhere to the bottom portion of a container, and can be loaded in a shape closer to a sphere, whereby the top portion of the formulation that has been loaded first often bulges out.

The shape and material of a container which is used for loading the cosmetic product of the present invention is also not at all limited as long as the container allows inner formulations to be viewed from the outside.

A container with any shape and material may be used as long as the container is a container used for common cosmetic products such as cream or gel. If the container is a shallow container, a formation loaded in the second phase contacts the bottom surface of the container and is loaded in a semispherical or semi-spheroid shape depending on the amount of the formulation loaded in the second phase, but this would not impair the functionality or novelty of the present invention, or impair the aesthetics of the invented product, or have any effect on the completion of the invention. If the depth of the container is sufficiently deep or depending on the shape of the loading container, the formulation loaded in the second phase can be loaded in a completely spherical shape depending on the amount of the formulation. This enables the final invented product to have a slightly different outward appearance from the former.

### EXAMPLES

While the present invention is explained hereafter with reference to the Examples, the technical scope of the present invention is defined by the Claims, and is not limitedly interpreted by the Examples below.

### Comparative Example 1

The following prescriptions were made based on Patent Literature 3 (Japanese Patent No. 5670031) as Comparative Example 1. The specific manufacturing method is as follows.

### (Prescription 1-1: Outer Layer Gel)

The components and the preparation method of the outer layer gel of Prescription 1-1 are as follows.

(A) Carboxyvinyl polymer: 0.4 (% by weight)
(A) Glycerin: 15 (% by weight)
(A) 1,3-butylene glycol: 15 (% by weight)
(A) Methyl parahydroxybenzoate: 0.15 (% by weight)
(A) Purified water: added as the remainder so that the total amount would be 100 (% by weight)
(B) Potassium hydroxide: 0.12 (% by weight)

After heating and dissolving component (A) while stirring, component (C) dissolved to be 10% using purified water was added. The mixture was then cooled to room temperature while stirring, resulting in a transparent gel.

### (Prescription 1-2: Inner Layer Cream)

The components and the preparation method of the inner layer cream of Prescription 1-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 2 (% by weight)
(A) Lipophilic glyceryl monostearate: 2.5 (% by weight)
(A) Methyl parahydroxybenzoate: 0.2 (i by weight)
(A) Natural vitamin E: 0.1 (% by weight)
(A) Hydrogenated soybean phospholipid: 0.3 (% by weight)
(B) Carboxyvinyl polymer: 0.5 (% by weight)
(B) Trehalose: 0.2 (% by weight)
(B) 1,3-butylene glycol: 5 (% by weight)
(B) Maltitol hydroxyalkyl(12,14) ether liquid: 0.3 (% by weight)
(B) Ethanol: 0.04 (% by weight)
(B) N-stearoyl-L-sodium glutamate: 0.5 (% by weight)
(B) Purified water: added as the remainder so that the total amount would be 100 (% by weight)
(C) Potassium hydroxide: 0.18 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 1-1 (outer layer part/bilayer gel cream) and the cream formulation of Prescription 1-2 (inner layer part/bilayer gel cream) were prepared by the above method, 100 g thereof was placed in a 100 ml beaker and was left standing at room temperature for 24 hours to measure the maximum load. The physical property value of each formulation is as follows.

* Gel formulation of Prescription 1-1: maximum load 0.52 N
* Cream formulation of Prescription 1-2: maximum load 0.36 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

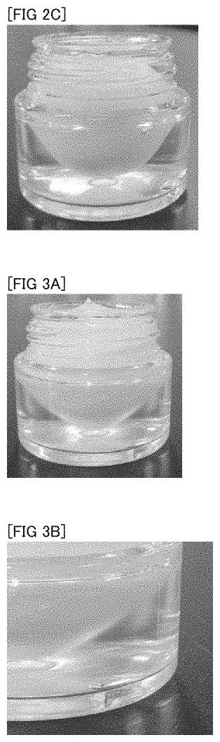

28 g of the gel of Prescription 1-1 was loaded in a transparent jar container with a total volume of 42 g using a syringe. The tip of a loading nozzle was disposed at the portion of two third of the entire amount from the bottom portion of the loaded gel, and 14 g of the emulsified composition of Prescription 1-2 was loaded, resulting in the bilayer gel cream shown in the photographs of FIG. 2A to FIG. 2C. The cream part of three among ten bilayer gel creams prepared by this method was loaded in a shape similar to a cone as shown in FIG. 2A and FIG. 2B. Unevenness was observed on the surface of the inner layer cream of all bilayer gel creams, which lacks aesthetics and production efficiency.

### (Confirmation of the Stability of Bilayer Gel Cream)

The gel cream after loading was left standing at 25° C. for 90 days to observe the outward appearance after 90 days. Measurement of the size of the cream formulation in the inner layer part of the bilayer gel cream indicates that the diameter of the cream part as of immediately after manufacturing expanded from 28 mm to 32 mm, and cracks were found in places on the surface of the cream. Further, white turbidity, which was not observed immediately after the preparation, was generated in the outer peripheral part of the inner layer cream after 10 days from the preparation of the bilayer gel cream. The level of the turbidity increased as the time passed, which significantly lacked stability. The photos are shown in FIG. 3A to FIG. 3C. As shown in the photos, unevenness and cracks were generated on the surface of the inner layer cream of the bilayer gel cream prepared by combining the outer layer gel of Prescription 1-1 and the inner layer cream of Prescription 1-2 as the inner layer cream expanded. Further, the bilayer gel cream also lacks stability under the storage condition at 40° C. for 6 months, such as generation of turbidity in the boundary surface between the cream and the gel after 5 days. This result indicates that the bilayer gel cream prepared by the method derived from the description of Patent Literature 3 (Japanese Patent No. 5670031) is incomplete in both stability and aesthetics, and needs to be further improved.

### Example 1

A plurality of prescriptions in which the prescriptions of Comparative Example 1 were changed were made. Among those prescriptions, one of the prescriptions that exhibited a good result was selected as Example 1. The specific manufacturing method is as follows.

### (Prescription 2-1: Outer Layer Gel)

The components and the preparation method of the outer layer gel of Prescription 2-1 are as follows.

(A) Acrylic acid/alkylmethacrylic acid copolymer: 0.7 (% by weight)
(A) Concentrated glycerin: 15 (% by weight)
(A) 1,3-butylene glycol: 5 (% by weight)
(A) Methyl parahydroxybenzoate: 0.15 (% by weight)
(A) Purified water: added as the remainder so that the total amount would be 100 (% by weight)
(B) Potassium hydroxide: 0.26 (% by weight)

After heating and dissolving component (A) while stirring, component (B) dissolved be to 10% using purified water was added. The mixture was then cooled to room temperature while stirring, resulting in a transparent gel.

### (Prescription 2-2: Inner Layer Cream)

The components and the preparation method of the inner layer cream of Prescription 2-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Propyl parahydroxybenzoate: 0.2 (% by weight)
(A) Natural vitamin E: 0.1 (% by weight)
(A) Hydrogenated soybean phospholipid: 0.3 (% by weight)
(B) Acrylic acid/alkylmethacrylic acid copolymer: 0.23 (% by weight)
(B) Concentrated glycerin: 10 (% by weight)
(B) 1,3-butylene glycol: 5 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Methyl parahydroxybenzoate: 0.2 (% by weight)
(B) Purified water: added as the remainder so that the total amount would be 100 (% by weight)
(C) Potassium hydroxide: 0.081 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 2-1 (outer layer part/bilayer gel cream) and the cream formulation of Prescription 2-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 2-1: maximum load 0.63 N
* Cream formulation of Prescription 2-2: maximum load 1.02 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

28 g of the gel of Prescription 2-1 was loaded in a transparent jar container with a total volume of 42 g using a syringe. The tip of a loading nozzle was disposed at the portion of two third of the entire amount from the bottom portion of the loaded gel, and 14 g of the emulsified composition of Prescription 2-2 was loaded, resulting in the bilayer gel cream shown in the photograph of FIG. 4.

### (Confirmation of the Stability of Bilayer Gel Cream)

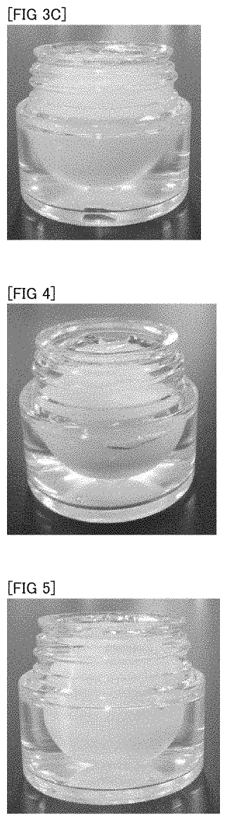

The gel cream after loading was left standing at 25° C. for 90 days to observe the outward appearance after 90 days. The photograph showing the result is shown as FIG. 5.

The size of the cream formulation in the inner layer part of the bilayer gel cream was measured after 90 days, and no change was observed in both the size and the outward appearance of the cream part as of immediately after manufacturing. This is also evident from the comparison between the photograph of FIG. 4 and the photograph of FIG. 5. Further, no turbidity was generated on the boundary surface between the cream and the gel, and there was no change in the outward appearance of the bilayer gel cream even after long-term storage at 40° C. for 6 months.

### Example 2

A plurality of prescriptions in which the prescriptions of Comparative Example 1 were changed were made. Among those prescriptions, another of the prescriptions that exhibited a good result was selected as Example 2. The specific manufacturing method is as follows.

### (Prescription 3-1: Outer Layer Gel)

The components and the preparation method of the outer layer gel of Prescription 3-1 are as follows.

(A) Acrylic acid/alkylmethacrylic acid copolymer: 0.7 (% by weight)
(A) Concentrated glycerin: 15 (% by weight)
(A) 1,3-butylene glycol: 5 (% by weight)
(A) Methyl parahydroxybenzoate: 0.15 (% by weight)
(A) Phenoxyethanol: 0.3 (% by weight)
(A) Purified water: added as the remainder so that the
total amount would be 100 (% by weight)
(B) Potassium hydroxide: 0.26 (% by weight)

After heating and dissolving component (A) while stirring, component (B) dissolved be to 10% using purified water was added. The mixture was then cooled to room temperature while stirring, resulting in a transparent gel.

### (Prescription 3-2: Inner Layer Cream)

The components and the preparation method of the inner layer cream of Prescription 3-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Propyl parahydroxybenzoate: 0.2 (% by weight)
(A) Natural vitamin E: 0.1 (% by weight)
(A) Hydrogenated soybean phospholipid: 0.1 (% by weight)
(B) Acrylic acid/alkylmethacrylic acid copolymer: 0.23 (% by weight)
(B) Xanthan gum: 0.1 (by weight)
(B) Concentrated glycerin: 10 (% by weight)
(B) 1,3-butylene glycol: 5 (% by weight)
(B) Phenoxyethanol: 0.3 (% by weight)
(B) N-stearoyl-L-sodium glutamate: 0.8 (% by weight)
(B) Propyl parahydroxybenzoate: 0.2 (% by weight)
(B) Purified water: added as the remainder so that the total amount would be 100 (% by weight)
(C) Potassium hydroxide: 0.081 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

(Measurement of physical property values)

After the gel formulation of Prescription 3-1 (outer layer part/bilayer gel cream) and the cream formulation of Prescription 3-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 3-1: maximum load 0.83 N
* Cream formulation of Prescription 3-2: maximum load 0.88 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

28 g of the gel of Prescription 3-1 was loaded in a transparent jar container with a total volume of 42 g using a syringe. The tip of a loading nozzle was disposed at the portion of two third of the entire amount from the bottom portion of the loaded gel, and 14 g of the emulsified composition of Prescription 3-2 was loaded, resulting in the bilayer gel cream shown in the photograph of FIG. 6.

(Confirmation of the Stability of Bilayer Gel Cream) The gel cream after loading was left standing at 25° C. for 90 days to observe the outward appearance after 90 days. The photograph showing the result is shown as FIG. 7.

The size of the cream formulation in the inner layer part of the bilayer gel cream was measured after 90 days, and no change was observed in both the size and the outward appearance of the cream part as of immediately after manufacturing. This is also evident from the comparison between the photograph of FIG. 6 and the photograph of FIG. 7. Further, no turbidity was generated on the boundary surface between the cream and the gel, and there was no change in the outward appearance of the bilayer gel cream even after long-term storage at 40° C. for 6 months.

### Example 3

A plurality of prescriptions in which the prescriptions of Comparative Example 1 were changed were made. Among those prescriptions, another of the prescriptions that exhibited a good result was selected as Example 3. The specific manufacturing method is as follows.

### (Prescription 4-1: Outer Layer Gel)

The components and the preparation method of the outer layer gel of Prescription 4-1 are as follows.

(A) Acrylic acid/alkylmethacrylic acid copolymer: 0.7 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)
(B) Potassium hydroxide: 0.26 (% by weight)

After heating and dissolving component (A) while stirring, 10% of component (B) dissolved in purified water was added. The mixture was then cooled to room temperature while stirring, resulting in a transparent gel.

### (Prescription 4-2: Inner Layer Cream)

The components and the preparation method of the inner layer cream of Prescription 4-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (t by weight)
(A) Natural vitamin E: 0.1 (% by weight)
(A) Hydrogenated soybean phospholipid: 0.1 (% by weight)
(B) Acrylic acid/alkylmethacrylic acid copolymer: 0.23 (% by weight)
(B) Xanthan gum: 0.1 (% by weight)
(B) Concentrated glycerin: 10 (% by weight)
(B) 1,3-butylene glycol: 1 (% by weight)
(B) 1,2-pentanediol: 4 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)
(C) Potassium hydroxide: 0.081 (% by weight)

After component (A) and component (B) were separately heated and mixed at 80° C., component (C) dissolved to be 10% using purified water was added was added. The mixture was then stirred using a homomixer. The mixture was stirred while being cooled to room temperature, resulting in a white and cream-like emulsified composition.

### (Measurement of Physical Property Values)

After the gel formulation of Prescription 4-1 (outer layer part/bilayer gel cream) and the cream formulation of Prescription 4-2 (inner layer part/bilayer gel cream) were prepared by the above method, 100 g thereof was placed in a 100 ml beaker and was left standing at room temperature for 24 hours to measure the maximum load and specific gravity.

The physical property value of each formulation is as follows.

* Gel formulation of Prescription 4-1: maximum load 0.76 N
* Cream formulation of Prescription 4-2: maximum load 0.75 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

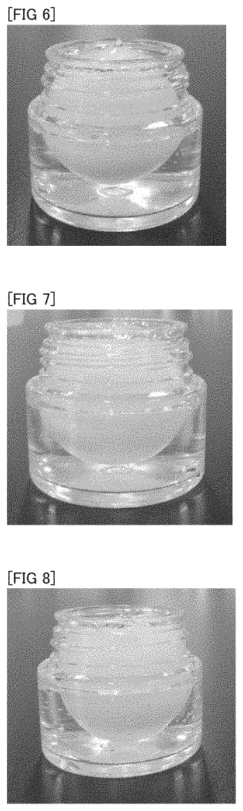

28 g of the gel of Prescription 4-1 was loaded in a transparent jar container with a total volume of 42 g using a syringe. The tip of a loading nozzle was disposed at the portion of two third of the entire amount from the bottom portion of the loaded gel, and 14 g of the emulsified composition of Prescription 4-2 was loaded, resulting in the bilayer gel cream shown in the photograph of FIG. 8.

### (Confirmation of the Stability of Bilayer Gel Cream)

The gel cream after loading was left standing at 25° C. for 90 days to observe the outward appearance after 90 days. The photograph showing the result is shown as FIG. 9.

The size of the cream formulation in the inner layer part of the bilayer gel cream was measured after 90 days, and no change was observed in both the size and the outward appearance of the cream part as of immediately after manufacturing. This is also evident from the comparison between the photograph of FIG. 8 and the photograph of FIG. 9. Further, no turbidity was generated on the boundary surface between the cream and the gel, and there was no change in the outward appearance of the bilayer gel cream even after long-term storage at 40° C. for 6 months.

### Example 4

A plurality of prescriptions in which the prescriptions of Example 3 were changed were made to evaluate the suitability of the formulation. One example was selected as Example 4. The specific manufacturing method is as follows.

### (Prescription 5-1: Outer Layer Gel)

The components and the preparation method of the outer layer gel of Prescription 5-1 are as follows.

(A) Acrylic acid/alkylmethacrylic acid copolymer: 0.7 (i by weight)
(B) Potassium hydroxide: 0.26 (% by weight)
(A) Sorbit liquid: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

After heating and dissolving component (A) while stirring, 10% of component (B) dissolved in purified water was added. The mixture was then cooled to room temperature while stirring, resulting in a transparent gel.

### (Prescription 5-2: Inner Layer Cream)

The components and the preparation method of the inner layer cream of Prescription 5-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (by weight)
(C) Potassium hydroxide: 0.081 (% by weight)
(B) Sorbit liquid: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (i by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 5-1 (outer layer part/bilayer gel cream) and the cream formulation of Prescription 5-2 (inner layer part/bilayer gel cream) were prepared by the above method, 100 g thereof was placed in a 100 ml beaker and was left standing at room temperature for 24 hours to measure the maximum load and specific gravity.

The physical property value of each formulation is as follows.

* Gel formulation of Prescription 5-1: maximum load 0.87 N
* Cream formulation of Prescription 5-2: maximum load 1.05 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

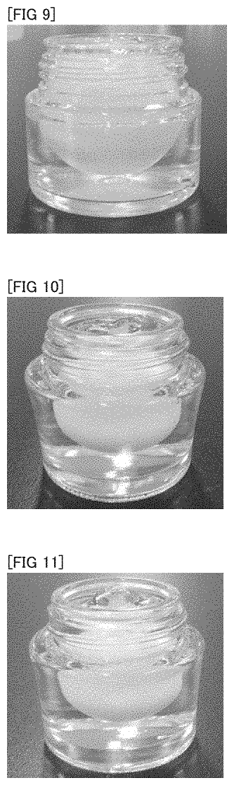

22 g of the gel of Prescription 5-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 5-2 was loaded, resulting in the bilayer gel cream shown in the photograph of FIG. 10.

### (Confirmation of the Stability of Bilayer Gel Cream)

The gel cream after loading was left standing at 25° C. for 90 days to observe the outward appearance after 90 days. The photograph showing the result is shown as FIG. 11.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 10 and the photograph of FIG. 11. Further, no turbidity was generated on the boundary surface between the cream and the gel, and there was no change in the outward appearance of the bilayer gel cream even after long-term storage at 40° C. for 6 months.

The components combined in Prescription 5-1 and Prescription 5-2 differ from those of Prescription 4-1 and Prescription 4-2 only in that concentrated glycerin added to the latter was changed to sorbit liquid. Since this change of design did not cause a change in the stability of the bilayer gel cream, it was revealed that changing a moisturizing agent from concentrated glycerin to sorbit liquid or the like does not impair the stability of a product.

### Example 5

A plurality of prescriptions in which the prescriptions of Example 3 were changed were made to evaluate the suitability of the formulation. One example was selected as Example 5. The specific manufacturing method is as follows.

### (Prescription 6-1: Outer Layer Gel)

The components and the preparation method of the outer layer gel of Prescription 6-1 are as follows.

(A) Acrylic acid/alkylmethacrylic acid copolymer: 0.7 (% by weight)
(B) Potassium hydroxide: 0.26 (% by weight)
(A) Dipropylene glycol: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

After heating and dissolving component (A) while stirring, 10% of component (B) dissolved in purified water was added. The mixture was then cooled to room temperature while stirring, resulting in a transparent gel.

### (Prescription 6-2: Inner Layer Cream)

The components and the preparation method of the inner layer cream of Prescription 6-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) Dipropylene glycol: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 6-1 (outer layer part/bilayer gel cream) and the cream formulation of Prescription 6-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 6-1: maximum load 0.66 N
* Cream formulation of Prescription 6-2: maximum load 1.27 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 6-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 6-2 was loaded, resulting in the bilayer gel cream shown in the photograph of FIG. 12.

### (Confirmation of the Stability of Bilayer Gel Cream)

The gel cream after loading was left standing at 25° C. for 90 days to observe the outward appearance after 90 days. The photograph showing the result is shown as FIG. 13.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 12 and the photograph of FIG. 13. Further, no turbidity was generated on the boundary surface between the cream and the gel, and there was no change in the outward appearance of the bilayer gel cream even after long-term storage at 40° C. for 6 months.

The components combined in Prescription 6-1 and Prescription 6-2 differ from those of Prescription 4-1 and Prescription 4-2 only in that concentrated glycerin added to the latter was changed to dipropylene glycol. Since this change of design did not cause a change in the stability of the bilayer gel cream, it was revealed that changing a moisturizing agent from concentrated glycerin to dipropylene glycol or the like does not impair the stability of a product.

### Example 6

A plurality of prescriptions in which the prescriptions of Example 3 were changed were made to evaluate the suitability of the formulation. One example was selected as Example 6. The specific manufacturing method is as follows.

### (Prescription 7-1: Outer Layer Gel)

The components and the preparation method of the outer layer gel of Prescription 7-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,2-pentanediol: 4 (% by weight)
(A) Purified water: added as the remainder so that the total amount would be 100 (% by weight)

### (Prescription 7-2: Inner Layer Cream)

The components and the preparation method of the inner layer cream of Prescription 7-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) Concentrated glycerin: 10 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 7-1 (outer layer part/bilayer gel cream) and the cream formulation of Prescription 7-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 7-1: maximum load 0.76 N
* Cream formulation of Prescription 7-2: maximum load 0.96 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

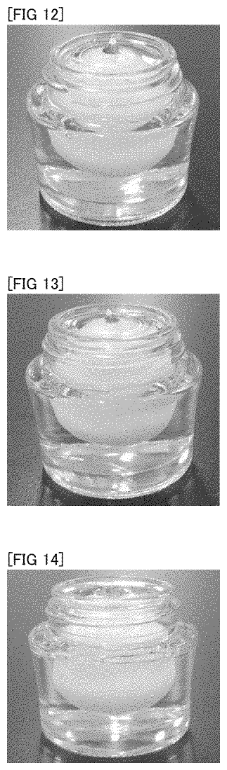

22 g of the gel of Prescription 7-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 7-2 was loaded, resulting in the bilayer gel cream shown in the photograph of FIG. 14.

### (Confirmation of the Stability of Bilayer Gel Cream)

The gel cream after loading was left standing at 25° C. for 90 days to observe the outward appearance after 90 days. The photograph showing the result is shown as FIG. 15.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 14 and the photograph of FIG. 15. Further, no turbidity was generated on the boundary surface between the cream and the gel, and there was no change in the outward appearance of the bilayer gel cream even after long-term storage at 40° C. for 6 months.

The components combined in Prescription 7-1 and Prescription 7-2 differ from those of Prescription 4-1 and Prescription 4-2 only in that 1,3-butylene glycol added to the latter was changed to purified water. Since this change of design did not cause a change in the stability of the bilayer gel cream, it was revealed that the presence or absence of addition of 1,3-butylene glycol does not change the stability of a product.

### Example 7

A plurality of prescriptions in which the prescriptions of Example 3 were changed were made to evaluate the suitability of the formulation. One example was selected as Example 7. The specific manufacturing method is as follows.

### (Prescription 8-1: Outer Layer Gel)

The components and the preparation method of the outer layer gel of Prescription 8-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

### (Prescription 8-2: Inner Layer Cream)

The components and the preparation method of the inner layer cream of Prescription 8-2 are as follows.
(A) Squalane: 11 (by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 8-1 (outer layer part/bilayer gel cream) and the cream formulation of Prescription 8-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 8-1: maximum load 0.78 N
* Cream formulation of Prescription 8-2: maximum load 1.24 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 8-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 8-2 was loaded, resulting in the bilayer gel cream shown in the photograph of FIG. 16.

### (Confirmation of the Stability of Bilayer Gel Cream)

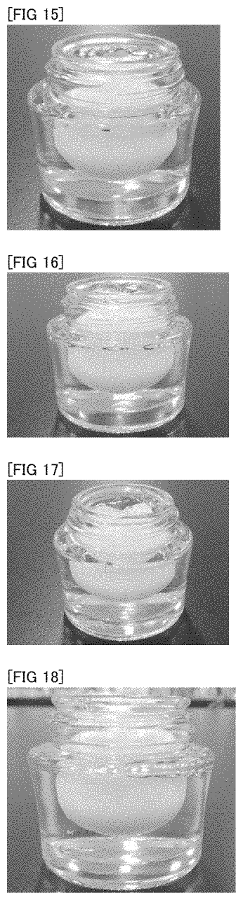

The gel cream after loading was left standing at 25° C. for 90 days to observe the outward appearance after 90 days. The photograph showing the result is shown as FIG. 17.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 16 and the photograph of FIG. 17. Further, slight turbidity was generated in the outer peripheral part of the inner layer cream, that is, the boundary surface between the inner layer cream and the outer layer gel, after 1 month under long-term storage at 40° C.

The components combined in the inner layer cream of Prescription 8-2 differ from those of Prescription 4-2 only in that glyceryl tri-2-ethylhexanoate added to the latter was changed to squalane. Since this change of design slightly deteriorated the stability of the bilayer gel cream upon the long-term storage test at 40° C., glyceryl tri-2-ethylhexanoate is more desirable than squalane as oil of the inner layer cream.

### Example 8

A plurality of prescriptions in which the prescriptions of Example 3 were changed were made to evaluate the suitability of the formulation. One example was selected as Example 8. The specific manufacturing method is as follows.

### (Prescription 9-1: Outer Layer Gel)

The components and the preparation method of the outer layer gel of Prescription 9-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

### (Prescription 9-2: Inner Layer Cream)

The components and the preparation method of the inner layer cream of Prescription 9-2 are as follows.

(A) Olive oil: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (t by weight)
(A) Natural vitamin E: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 9-1 (outer layer part/bilayer gel cream) and the cream formulation of Prescription 9-2 (inner layer part/bilayer gel cream) were prepared by the above method, 100 g thereof was placed in a 100 ml beaker and was left standing at room temperature for 24 hours to measure the maximum load and specific gravity.

The physical property value of each formulation is as follows.

* Gel formulation of Prescription 9-1: maximum load 0.78 N
* Cream formulation of Prescription 9-2: maximum load 1.07 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

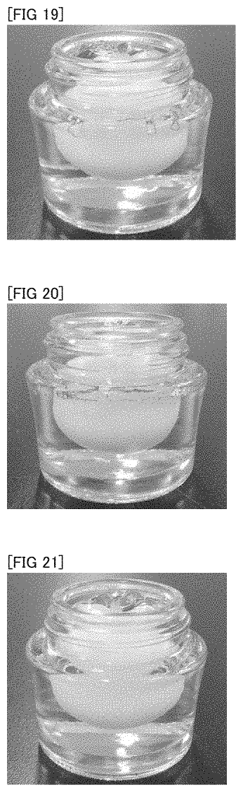

22 g of the gel of Prescription 9-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 9-2 was loaded, resulting in the bilayer gel cream shown in the photograph of FIG. 18.

### (Confirmation of the Stability of Bilayer Gel Cream)

The gel cream after loading was left standing at 25° C. for 90 days to observe the outward appearance after 90 days. The photograph showing the result is shown as FIG. 19.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 18 and the photograph of FIG. 19. Further, slight turbidity was generated in the outer peripheral part of the inner layer cream, that is, the boundary surface between the inner layer cream and the outer layer gel, after 1 month under long-term storage at 40° C.

The components combined in the inner layer cream of Prescription 9-2 differ from those of Prescription 4-2 only in that glyceryl tri-2-ethylhexanoate added to the latter was changed to olive oil. Since this change of design slightly deteriorated the stability of the bilayer gel cream upon the long-period storage test at 40° C., glyceryl tri-2-ethylhexanoate is more desirable than olive oil as oil of the inner layer cream.

### Example 9

A plurality of prescriptions in which the prescriptions of Example 3 were changed were made to evaluate the suitability of the formulation. One example was selected as Example 9. The specific manufacturing method is as follows.

### (Prescription 10-1: Outer Layer Gel)

The components and the preparation method of the outer layer gel of Prescription 10-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

### (Prescription 10-2: Inner Layer Cream)

The components and the preparation method of the inner layer cream of Prescription 10-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 10-1 (outer layer part/bilayer gel cream) and the cream formulation of Prescription 10-2 (inner layer part/bilayer gel cream) were prepared by the above method, 100 g thereof was placed in a 100 ml beaker and was left standing at room temperature for 24 hours to measure the maximum load and specific gravity.

The physical property value of each formulation is as follows.

* Gel formulation of Prescription 10-1: maximum load 0.78 N
* Cream formulation of Prescription 10-2: maximum load 1.18 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 10-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 10-2 was loaded, resulting in the bilayer gel cream shown in the photograph of FIG. 20.

### (Confirmation of the Stability of Bilayer Gel Cream)

The gel cream after loading was left standing at 25° C. for 90 days to observe the outward appearance after 90 days. The photograph showing the result is shown as FIG. 21.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 20 and the photograph of FIG. 21. Further, no turbidity was generated on the boundary surface between the cream and the gel, and there was no change in the outward appearance of the bilayer gel cream even after long-term storage at 40° C. for 6 months.

Regarding the components combined in Prescription 10-1 and Prescription 10-2, Prescription 10-1 is the same as Prescription 4-1, and Prescription 10-2 differs from Prescription 4-2 only in that natural vitamin E added to the latter was changed to purified water. Since this change of design did not cause a change in the stability of the bilayer gel cream, it was revealed that the presence or absence of addition of natural vitamin E does not change the stability of a product.

### Example 10

A plurality of prescriptions in which the prescriptions of Example 3 were changed were made to evaluate the suitability of the formulation. One example was selected as Example 10. The specific manufacturing method is as follows.

### (Prescription 11-1: Outer Layer Gel)

The components and the preparation method of the outer layer gel of Prescription 11-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

### (Prescription 11-2: Inner Layer Cream)

The components and the preparation method of the inner layer cream of Prescription 11-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Dibutylhydroxytoluene: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 11-1 (outer layer part/bilayer gel cream) and the cream formulation of Prescription 11-2 (inner layer part/bilayer gel cream) were prepared by the above method, 100 g thereof was placed in a 100 ml beaker and was left standing at room temperature for 24 hours to measure the maximum load and specific gravity.

The physical property value of each formulation is as follows.

* Gel formulation of Prescription 11-1: maximum load 0.78 N
* Cream formulation of Prescription 11-2: maximum load 1.11 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

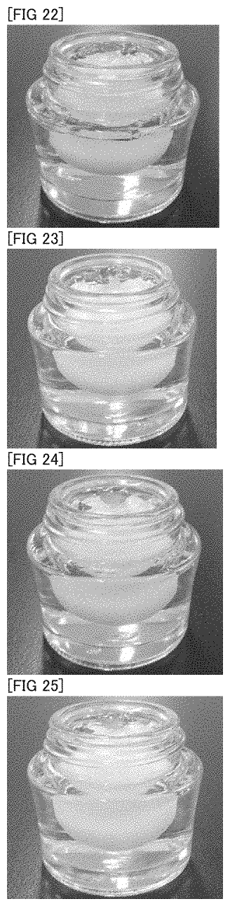

22 g of the gel of Prescription 11-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 11-2 was loaded, resulting in the bilayer gel cream shown in the photograph of FIG. 22.

### (Confirmation of the Stability of Bilayer Gel Cream)

The gel cream after loading was left standing at 25° C. for 90 days to observe the outward appearance after 90 days. The photograph showing the result is shown as FIG. 23.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 22 and the photograph of FIG. 23. Further, no turbidity was generated on the boundary surface between the cream and the gel, and there was no change in the outward appearance of the bilayer gel cream even after long-term storage at 40° C. for 6 months.

Regarding the components combined in Prescription 11-1 and Prescription 11-2, Prescription 11-1 is the same as Prescription 4-1, and Prescription 11-2 differs from Prescription 4-2 only in that natural vitamin E added to the latter was changed to dibutylhydroxytoluene. Since this change of design did not cause a change in the stability of the bilayer gel cream, it was revealed that changing an antioxidant from natural vitamin E to dibutylhydroxytoluene or the like does not impair the stability of a product.

### Example 11

A plurality of prescriptions in which the prescriptions of Example 3 were changed were made to evaluate the suitability of the formulation. One example was selected as Example 11. The specific manufacturing method is as follows.

### (Prescription 12-1: Outer Layer Gel)

The components and the preparation method of the outer layer gel of Prescription 12-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

### (Prescription 12-2: Inner Layer Cream)

The components and the preparation method of the inner layer cream of Prescription 12-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (i by weight)
(A) Lipophilic glyceryl monostearate: 2.5 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(C) Potassium hydroxide: 0.081 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 12-1 (outer layer part/bilayer gel cream) and the cream formulation of Prescription 12-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 12-1: maximum load 0.78 N
* Cream formulation of Prescription 12-2: maximum load 1.36 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 12-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 12-2 was loaded, resulting in the bilayer gel cream shown in the photograph of FIG. 24.

### (Confirmation of the Stability of Bilayer Gel Cream)

The gel cream after loading was left standing at 25° C. for 90 days to observe the outward appearance after 90 days. The photograph showing the result is shown as FIG. 25.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 24 and the photograph of FIG. 25. Further, no turbidity was generated on the boundary surface between the cream and the gel, and there was no change in the outward appearance of the bilayer gel cream even after long-term storage at 40° C. for 6 months.

Regarding the components combined in Prescription 12-1 and Prescription 12-2, Prescription 12-1 is the same as Prescription 4-1, and Prescription 12-2 differs from Prescription 4-2 only in that xanthan gum added to the latter was changed to purified water. Since this change of design did not cause a change in the stability of the bilayer gel cream, it was revealed that the presence or absence of addition of xanthan gum does not change the stability of a product.

### Example 12

A plurality of prescriptions in which the prescriptions of Example 3 were changed were made to evaluate the suitability of the formulation. One example was selected as Example 12. The specific manufacturing method is as follows.

### (Prescription 13-1: Outer Layer Gel)

The components and the preparation method of the outer layer gel of Prescription 13-1 are as follows.

(A) Acrylic acid/alkylmethacrylic acid copolymer: 0.7 (% by weight)
(B) L-arginine: 0.55 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

### (Prescription 13-2: Inner Layer Cream)

The components and the preparation method of the inner layer cream of Prescription 13-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) L-arginine: 0.175 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 13-1 (outer layer part/bilayer gel cream) and the cream formulation of Prescription 13-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 13-1: maximum load 0.80 N
* Cream formulation of Prescription 13-2: maximum load 1.01 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

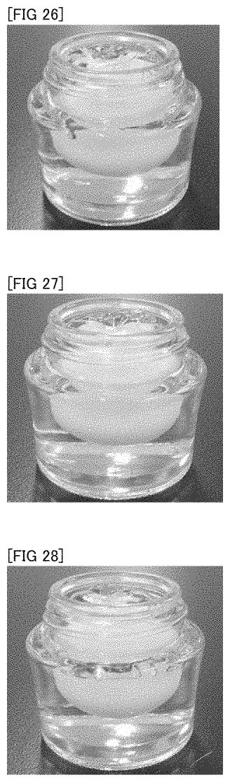

22 g of the gel of Prescription 13-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 13-2 was loaded, resulting in the bilayer gel cream shown in the photograph of FIG. 26.

### (Confirmation of the Stability of Bilayer Gel Cream)

The gel cream after loading was left standing at 25° C. for 90 days to observe the outward appearance after 90 days. The photograph showing the result is shown as FIG. 27.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 26 and the photograph of FIG. 27. Further, no turbidity was generated on the boundary surface between the cream and the gel, and there was no change in the outward appearance of the bilayer gel cream even after long-term storage at 40° C. for 6 months.

The components combined in Prescription 13-1 and Prescription 13-2 differ from those of Prescription 4-1 and Prescription 4-2 only in that potassium hydroxide added to the latter was changed to L-arginine. Since this change of design did not cause a change in the stability of the bilayer gel cream, it was revealed that changing a neutralizing agent from potassium hydroxide to L-arginine or the like does not impair the stability of a product.

### Example 13

A plurality of prescriptions in which the prescriptions of Example 3 were changed were made to evaluate the suitability of the formulation. One example was selected as Example 13. The specific manufacturing method is as follows.

### (Prescription 14-1: Outer Layer Gel)

The components and the preparation method of the outer layer gel of Prescription 14-1 are as follows.

(A) Acrylic acid/alkylmethacrylic acid copolymer: 0.7 (% by weight)
(B) Triethanolamine: 0.5 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

### (Prescription 14-2: Inner Layer Cream)

The components and the preparation method of the inner layer cream of Prescription 14-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) Triethanolamine: 0.15 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 14-1 (outer layer part/bilayer gel cream) and the cream formulation of Prescription 14-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 14-1: maximum load 0.85 N
* Cream formulation of Prescription 14-2: maximum load 0.97 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 14-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 14-2 was loaded, resulting in the bilayer gel cream shown in the photograph of FIG. 28.

### (Confirmation of the Stability of Bilayer Gel Cream)

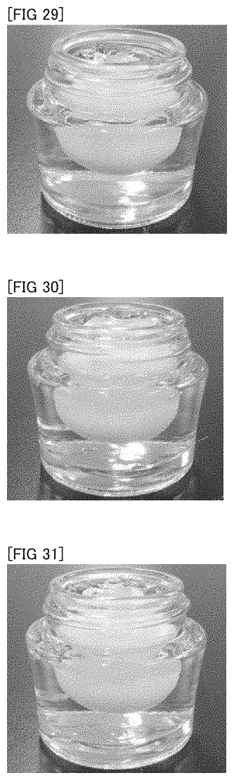

The gel cream after loading was left standing at 25° C. for 90 days to observe the outward appearance after 90 days. The photograph showing the result is shown as FIG. 29.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 28 and the photograph of FIG. 29. Further, no turbidity was generated on the boundary surface between the cream and the gel, and there was no change in the outward appearance of the bilayer gel cream even after long-term storage at 40° C. for 6 months.

The components combined in Prescription 14-1 and Prescription 14-2 differ from those of Prescription 4-1 and Prescription 4-2 only in that potassium hydroxide added to the latter was changed to triethanolamine. Since this change of design did not cause a change in the stability of the bilayer gel cream, it was revealed that changing a neutralizing agent from potassium hydroxide to triethanolamine or the like does not impair the stability of a product.

### Example 14

A plurality of prescriptions in which the prescriptions of Example 3 were changed were made to evaluate the suitability of the formulation. One example was selected as Example 14. The specific manufacturing method is as follows.

### (Prescription 15-1: Outer Layer Gel)

The components and the preparation method of the outer layer gel of Prescription 15-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 1 (% by weight)
(A) 1,2-pentanediol: 3 (% by weight)
(A) Purified water: added as the remainder so that the total amount would be 100 (% by weight)

### (Prescription 15-2: Inner Layer Cream)

The components and the preparation method of the inner layer cream of Prescription 15-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 3 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 15-1 (outer layer part/bilayer gel cream) and the cream formulation of Prescription 15-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 15-1: maximum load 0.87 N
* Cream formulation of Prescription 15-2: maximum load 0.94 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 15-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 15-2 was loaded, resulting in the bilayer gel cream shown in the photograph of FIG. 30.

### (Confirmation of the Stability of Bilayer Gel Cream)

The gel cream after loading was left standing at 25° C. for 90 days to observe the outward appearance after 90 days. The photograph showing the result is shown as FIG. 31.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 30 and the photograph of FIG. 31. Further, no turbidity was generated on the boundary surface between the cream and the gel, and there was no change in the outward appearance of the bilayer gel cream even after long-term storage at 40° C. for 6 months.

The components combined in Prescription 15-1 and Prescription 15-2 differ from those of Prescription 4-1 and Prescription 4-2 only in that the amount of 1,2-pentanediol added to the latter was changed from 4% to 3%. Since this change of design did not cause a change in the stability of the bilayer gel cream, it was revealed that changing the amount of added 1,2-pentanediol from 4% to 3% does not impair the stability of a product.

### Example 15

A plurality of prescriptions in which the prescriptions of Example 3 were changed were made to evaluate the suitability of the formulation. One example was selected as Example 15. The specific manufacturing method is as follows.

### (Prescription 16-1: Outer Layer Gel)

The components and the preparation method of the outer layer gel of Prescription 16-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 1 (% by weight)
(A) 1,2-pentanediol: 5 (% by weight)
(A) Purified water: added as the remainder so that the total amount would be 100 (% by weight)

### (Prescription 16-2: Inner Layer Cream)

The components and the preparation method of the inner layer cream of Prescription 16-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 5 (% by weight)
(B) Acrylic acid/alkylmethacrylic acid copolymer: 0.23 (% by weight)
(B) Xanthan gum: 0.1 (by weight)
(C) Potassium hydroxide: 0.081 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 16-1 (outer layer part/bilayer gel cream) and the cream formulation of Prescription 16-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 16-1: maximum load 0.85 N
* Cream formulation of Prescription 16-2: maximum load 1.27 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 16-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 16-2 was loaded, resulting in the bilayer gel cream shown in the photograph of FIG. 32.

### (Confirmation of the Stability of Bilayer Gel Cream)

The gel cream after loading was left standing at 25° C. for 90 days to observe the outward appearance after 90 days. The photograph showing the result is shown as FIG. 33.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 32 and the photograph of FIG. 33. Further, no turbidity was generated on the boundary surface between the cream and the gel, and there was no change in the outward appearance of the bilayer gel cream even after long-term storage at 40° C. for 6 months.

The components combined in Prescription 16-1 and Prescription 16-2 differ from those of Prescription 4-1 and Prescription 4-2 only in that the amount of 1,2-pentanediol added to the latter was changed from 4% to 5%. Since this change of design did not cause a change in the stability of the bilayer gel cream, it was revealed that changing the amount of added 1,2-pentanediol from 4% to 5% does not impair the stability of a product.

### Example 16

A plurality of prescriptions in which the prescriptions of Example 3 were changed were made to evaluate the suitability of the formulation. One example was selected as Example 16. The specific manufacturing method is as follows.

### (Prescription 17-1: Outer Layer Gel)

The components and the preparation method of the outer layer gel of Prescription 17-1 are as follows.

(A) Acrylic acid/alkylmethacrylic acid copolymer: 0.7 (i by weight)
(B) Potassium hydroxide: 0.26 (% by weight)
(A) 1,3-butylene glycol: 1 (% by weight)
(A) 1,2-pentanediol: 4 (% by weight)
(A) Purified water: added as the remainder so that the total amount would be 100 (% by weight)

### (Prescription 17-2: Inner Layer Cream)

The components and the preparation method of the inner layer cream of Prescription 17-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 17-1 (outer layer part/bilayer gel cream) and the cream formulation of Prescription 17-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 17-1: maximum load 0.79 N
* Cream formulation of Prescription 17-2: maximum load 0.77 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

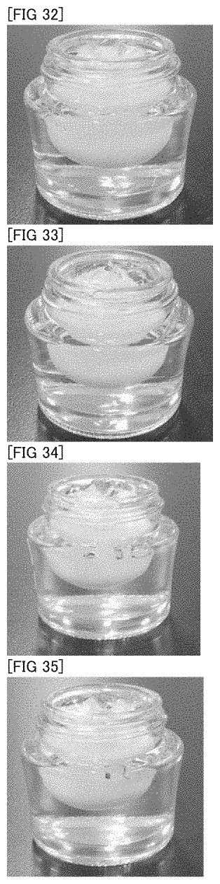

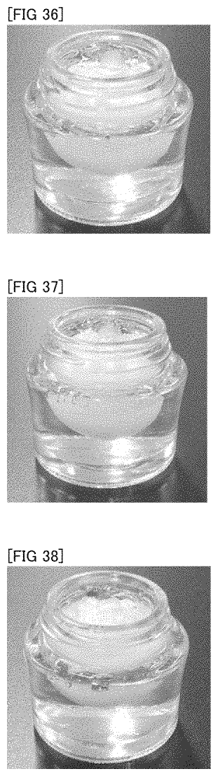

22 g of the gel of Prescription 17-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 17-2 was loaded, resulting in the bilayer gel cream shown in the photograph of FIG. 34.

### (Confirmation of the Stability of Bilayer Gel Cream)

The gel cream after loading was left standing at 25° C. for 90 days to observe the outward appearance after 90 days.

The photograph showing the result is shown as FIG. 35.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 34 and the photograph of FIG. 35. Further, no turbidity was generated on the boundary surface between the cream and the gel, and there was no change in the outward appearance of the bilayer gel cream even after long-term storage at 40° C. for 6 months.

The components combined in Prescription 17-1 and Prescription 17-2 differ from those of Prescription 4-1 and Prescription 4-2 only in that concentrated glycerin added to the latter was changed to purified water. Since this change of design did not cause a change in the stability of the bilayer gel cream, it was revealed that the presence or absence of addition of concentrated glycerin does not change the stability of a product.

### Example 17

A plurality of prescriptions in which the prescriptions of Example 3 were changed were made to evaluate the suitability of the formulation. One example was selected as Example 17. The specific manufacturing method is as follows.

### (Prescription 18-1: Outer Layer Gel)

The components and the preparation method of the outer layer gel of Prescription 18-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (i by weight)

### (Prescription 18-2: Inner Layer Cream)

The components and the preparation method of the inner layer cream of Prescription 18-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 4 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) 1,3-butylene glycol: 1 (% by weight)
(B) Concentrated glycerin: 10 (% by weight)
(B) Maltitol hydroxyalkyl(12,14) ether liquid: 0.3 (% by weight)
(B) Ethanol: 0.04 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 18-1 (outer layer part/bilayer gel cream) and the cream formulation of Prescription 18-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 18-1: maximum load 0.78 N
* Cream formulation of Prescription 18-2: maximum load 1.06 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 18-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 18-2 was loaded, resulting in the bilayer gel cream shown in the photograph of FIG. 36.

### (Confirmation of the Stability of Bilayer Gel Cream)

The gel cream after loading was left standing at 25° C. for 90 days to observe the outward appearance after 90 days. The photograph showing the result is shown as FIG. 37.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 36 and the photograph of FIG. 37. Further, no turbidity was generated on the boundary surface between the cream and the gel, and there was no change in the outward appearance of the bilayer gel cream even after long-term storage at 40° C. for 6 months.

The components combined in Prescription 18-1 are the same as those of Prescription 4-1. The components combined in Prescription 18-2 differ from those of Prescription 4-2 only in that maltitol laurate added to the latter was changed to maltitol hydroxyalkyl(12,14) ether liquid. Since this change of design did not cause a change in the stability of the bilayer gel cream, it was revealed that changing maltitol laurate to another emulsifying agent such as maltitol hydroxyalkyl(12,14) ether liquid does not change the stability of a product.

### Example 18

A plurality of prescriptions in which the prescriptions of Example 3 were changed were made to evaluate the suitability of the formulation. One example was selected as Example 18. The specific manufacturing method is as follows.

### (Prescription 19-1: Outer Layer Gel)

The components and the preparation method of the outer layer gel of Prescription 19-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 1 (% by weight)
(A) Methyl parahydroxybenzoate: 0.15 (% by weight)
(A) Purified water: added as the remainder so that the total amount would be 100 (i by weight)

### (Prescription 19-2: Inner Layer Cream)

The components and the preparation method of the inner layer cream of Prescription 19-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) Methyl parahydroxybenzoate: 0.2 (% by weight)
(B) Acrylic acid/alkylmethacrylic acid copolymer: 0.23 (% by weight)
(B) Xanthan gum: 0.1 (% by weight)
(C) Potassium hydroxide: 0.081 (% by weight)
(B) 1,3-butylene glycol: 1 (% by weight)
(B) Concentrated glycerin: 10 (% by weight)
(B) Maltitol laurate: 0.34 (% by weight)
(B) Dehydrated ethanol: 0.07 (% by weight)
(B) N-stearoyl-L-sodium glutamate: 0.5 (% by weight)
(B) Purified water: added as the remainder so that the total amount would be 100 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 19-1 (outer layer part/bilayer gel cream) and the cream formulation of Prescription 19-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 19-1: maximum load 0.88 N
* Cream formulation of Prescription 19-2: maximum load 0.88 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 19-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 19-2 was loaded, resulting in the bilayer gel cream shown in the photograph of FIG. 38.

### (Confirmation of the Stability of Bilayer Gel Cream)

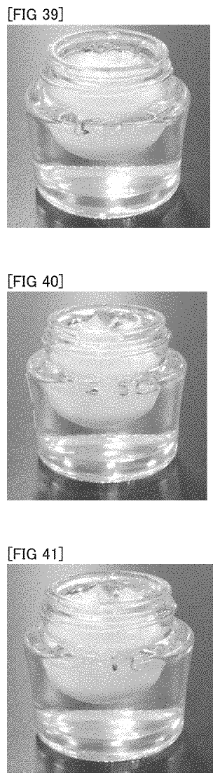

The gel cream after loading was left standing at 25° C. for 90 days to observe the outward appearance after 90 days. The photograph showing the result is shown as FIG. 39.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 38 and the photograph of FIG. 39. Further, no turbidity was generated on the boundary surface between the cream and the gel, and there was no change in the outward appearance of the bilayer gel cream even after long-term storage at 40° C. for 6 months.

The components combined in Prescription 19-1 and Prescription 19-2 differ from those of Prescription 4-1 and Prescription 4-2 only in that 1,2-pentanediol added to the latter was changed to methyl parahydroxybenzoate. Since this change of design did not cause a change in the stability of the bilayer gel cream, it was revealed that changing 1,2-pentanediol to a preservative such as methyl parahydroxybenzoate does not change the stability of a product.

### Example 19

A plurality of prescriptions in which the prescriptions of Example 3 were changed were made to evaluate the suitability of the formulation. One example was selected as Example 19. The specific manufacturing method is as follows.

### (Prescription 20-1: Outer Layer Gel)

The components and the preparation method of the outer layer gel of Prescription 20-1 are as follows.

(A) Carboxyvinyl polymer: 0.4 (% by weight)
(B) Potassium hydroxide: 0.12 (% by weight)
(A) Concentrated glycerin: 15 (% by weight)
(A) 1,3-butylene glycol: 1 (% by weight)
(A) 1,2-pentanediol: 4 (% by weight)
(A) Purified water: added as the remainder so that the total amount would be 100 (% by weight)

### (Prescription 20-2: Inner Layer Cream)

The components and the preparation method of the inner layer cream of Prescription 20-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 5 (% by weight)
(A) Cetanol: 1 (% by weight)
(A) Lipophilic glyceryl monostearate: 2.5 (% by weight)
(A) Natural vitamin E: 0.1 (% by weight)
(A) Hydrogenated soybean phospholipid: 0.1 (% by weight)
(B) 1,2-pentanediol: 4 (% by weight)
(B) Carboxyvinyl polymer: 0.5 (% by weight)
(B) Xanthan gum: 0.1 (% by weight)
(C) Potassium hydroxide: 0.18 (% by weight)
(B) Concentrated glycerin: 10 (% by weight)
(B) 1,3-butylene glycol: 1 (% by weight)
(B) Maltitol laurate: 0.34 (% by weight)
(B) Dehydrated ethanol: 0.07 (% by weight)
(B) N-stearoyl-L-sodium glutamate: 0.5 (by weight)
(B) Purified water: added as the remainder so that the total amount would be 100 (i by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 20-1 (outer layer part/bilayer gel cream) and the cream formulation of Prescription 20-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 20-1: maximum load 0.54 N
* Cream formulation of Prescription 20-2: maximum load 1.58 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

22 g of the gel of Prescription 20-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 20-2 was loaded, resulting in the bilayer gel cream shown in the photograph of FIG. 40.

### (Confirmation of the Stability of Bilayer Gel Cream)

The gel cream after loading was left standing at 25° C. for 90 days to observe the outward appearance after 90 days. The photograph showing the result is shown as FIG. 41.

The size of the cream formulation in the inner layer part of the bilayer gel cream was compared after 1 month, finding that the cream part as of immediately after manufacturing expanded and slight turbidity was generated on the boundary surface between the cream and gel. This is also evident from the comparison between the photograph of FIG. 40 and the photograph of FIG. 41. Further, this tendency was more significant in the long-term storage at 40° C. for 6 months.

The components combined in Prescription 20-1 and Prescription 20-2 differ from those of Prescription 4-1 and Prescription 4-2 only in that acrylic acid/alkylmethacrylic acid copolymer added to the latter was changed to carboxyvinyl polymer and the amount of added potassium hydroxide, which is a neutralizing agent, was adjusted so that appropriate pH can be maintained.

Since this change of design changed the stability of the bilayer gel cream, it was revealed that adding acrylic acid/alkylmethacrylic acid copolymer is an important element upon maintaining the dosage form of the present formulation.

### Example 20

A plurality of prescriptions in which the prescriptions of Example 3 were changed were made to evaluate the suitability of the formulation. One example was selected as Example 20. The specific manufacturing method is as follows.

### (Prescription 21-1: Outer Layer Gel)

The components and the preparation method of the outer layer gel of Prescription 21-1 are as follows.

(A) Acrylic acid/alkylmethacrylic acid copolymer: 0.7 (% by weight)
(B) Potassium hydroxide: 0.26 (% by weight)
(A) Concentrated glycerin: 15 (% by weight)
(A) 1,3-butylene glycol: 15 (% by weight)
(A) Methyl parahydroxybenzoate: 0.15 (% by weight)
(A) Purified water: added as the remainder so that the total amount would be 100 (% by weight)

### (Prescription 21-2: Inner Layer Cream)

The components and the preparation method of the inner layer cream of Prescription 18-2 are as follows.

(A) Glyceryl tri-2-ethylhexanoate: 11 (% by weight)
(A) Cetyl 2-ethylhexanoate: 5 (% by weight)
(A) Methyl polysiloxane: 0.5 (% by weight)
(A) Behenyl alcohol: 2 (% by weight)
(A) Lipophilic glyceryl monostearate: 2.5 (% by weight)
(A) Natural vitamin E: 0.1 (% by weight)
(A) Hydrogenated soybean phospholipid: 0.3 (% by weight)
(B) Methyl parahydroxybenzoate: 0.2 (% by weight)
(B) Xanthan gum: 0.1 (% by weight)
(B) Concentrated glycerin: 0.1 (% by weight)
(B) Acrylic acid/alkylmethacrylic acid copolymer: 0.23 (% by weight)
(B) 1,3-butylene glycol: 5 (% by weight)
(B) Maltitol hydroxyalkyl(12,14) ether liquid: 0.3 (r by weight)
(B) Ethanol: 0.04 (% by weight)
(B) N-stearoyl-L-sodium glutamate: 0.5 (% by weight)
(B) Purified water: added as the remainder so that the total amount would be 100 (i by weight)
(C) Potassium hydroxide: 0.081 (% by weight)

After component (A) and component (B) were separately heated and mixed at 80° C., component (C) dissolved to be 10% using purified water was added. The mixture was then stirred using a homomixer. The mixture was stirred while being cooled to room temperature, resulting in a white and cream-like emulsified composition.

### (Measurement of Physical Property Values)

After the gel formulation of Prescription 21-1 (outer layer part/bilayer gel cream) and the cream formulation of Prescription 21-2 (inner layer part/bilayer gel cream) were prepared by the above method, 100 g thereof was placed in a 100 ml beaker and was left standing at room temperature for 24 hours to measure the maximum load and specific gravity. The physical property value of each formulation is as follows.

* Gel formulation of Prescription 21-1: maximum load 0.90 N
* Cream formulation of Prescription 21-2: maximum load 0.43 N

The maximum load of each formulation was measured with SUN RHEO METER (COMPAC 100-II) purchased from Sun Scientific Co., Ltd., which measured the maximum value of load applied per cross-sectional area of a rod-like probe with a diameter of 20 mm when the rod-like probe is inserted 25 mm at a rate of 60 mm/min.

### (Method for Loading Bilayer Gel Cream)

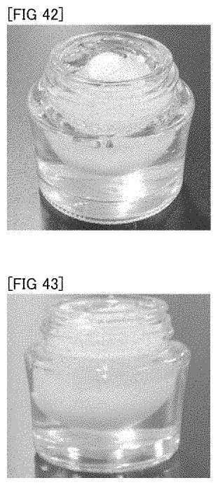

22 g of the gel of Prescription 21-1 was loaded in a transparent jar container with a total volume of 33 g using a syringe. The tip of a loading nozzle was disposed at the portion of two third of the entire amount from the bottom portion of the loaded gel, and 11 g of the emulsified composition of Prescription 21-2 was loaded, resulting in the bilayer gel cream shown in the photograph of FIG. 42.

### (Confirmation of the Stability of Bilayer Gel Cream)

The gel cream after loading was left standing at 25° C. for 90 days to observe the outward appearance after 90 days. The photograph showing the result is shown as FIG. 43.

The size of the cream formulation in the inner layer part of the bilayer gel cream was compared after 1 month, and no change was observed in both the size and the outward appearance of the cream part as of immediately after manufacturing. This is also evident from the comparison between the photograph of FIG. 42 and the photograph of FIG. 43. Further, no turbidity was generated on the boundary surface between the cream and the gel, and there was no change in the outward appearance of the bilayer gel cream even after long-term storage at 40° C. for 6 months.

The components combined in Prescription 21-1 and Prescription 21-2 differ from those of Prescription 1-1 and Prescription 1-2 only in that carboxyvinyl polymer added to the latter was changed to acrylic acid/alkylmethacrylic acid copolymer and the amount of added potassium hydroxide, which is a neutralizing agent, was adjusted so that appropriate pH can be maintained.

Since this change of design changed the stability of the bilayer gel cream, it was revealed that adding acrylic acid/alkylmethacrylic acid copolymer is an important element upon maintaining the dosage form of the present formulation.

### Example 21

Usage trials of the formulations made in Example 1 to Example 3 were conducted. In the usage trials, a user was instructed to take a suitable amount of formulation using a spatula and to apply the formulation to the skin. After use, the user was asked about the usability and functionality thereof in an interview format. The functionality of the formulation that can be confirmed from the trials, components that are added, and preparation method are listed below.

### (Functionality 1)

The bilayer gel cream of Examples 1 to 3 is a formulation that has the functionalities of both cream containing an oily component in abundance and beauty gel containing an aqueous component and a moisturizing component such as glycerin or 1,3-butylene glycol in abundance in a single formulation. When this formulation was taken with a spatula, including the formulation in the outer layer part and the inner formulation, and applied to the skin, the formulation had characteristic usability having both moisturizing feeling of beauty gel and emollient taste of cream. It was also confirmed that a high humectifying effect, emollient effect, and moisturizing effect on the skin could be felt.

### (Functionality 2)

The bilayer gel cream of Examples 1 to 3 consists of a beauty gel part containing a lot of aqueous components and a cream part containing a lot of oily components. In this manner, oily components and aqueous components are separated in a single formulation in the bilayer gel cream. Selectivity of components that can be added is thereby improved.

### (Functionality 3)

In the bilayer gel cream of Examples 1 to 3, two types of formulations are divided into the gel formulation in the outer layer part and the inner cream formulation, in which the amount of the inner cream formulation which is added is about one third of the entire amount. It was confirmed that, for the above reason, it is not necessary to disperse a surfactant in the entire formulations, and the amount of a surfactant necessary for emulsification can be reduced to about one third. Further, it was confirmed that the bilayer gel cream of Example 1 is a formulation having impression upon use in which the irritation to and burden on the skin due to a surfactant is drastically reduced.

### (Functionality 4)

The bilayer gel cream of Examples 1 to 3 was stored at temperature of 40° C., 50° C., 4° C., and 25° C. to observe the change in the state after 3 months. No change was recognized in the formulation at any temperature. It was confirmed from this result that the bilayer loaded gel cream of Example 1 is a formulation having high stability in spite of a moisturizing component such as glycerin or 1,3-butylene glycol being added to the outer layer part at high concentrations.

### (Functionality 5)

Two different types of formulations, i.e., highly transparent beauty gel in the outer layer part and inner cream, are combined in the bilayer gel cream of Examples 1 to 3. Through the usage trials, it was confirmed that it is possible to use the formulations while visually adjusting the amount of the two types of formulations to be used by using the formulations with a spatula. Specifically, it is possible to use a single formulation while making visual adjustments, such as using more of the highly transparent aqueous beauty gel in the daytime in which moisturizing components tend to be insufficient, and using more of the cream part containing more oil while sleeping or after washing the face, in which oil tends to be insufficient. In this manner, the bilayer gel cream can be used differently according to the skin condition despite being a single product.

### (Functionality 6)

In the bilayer gel cream of Examples 1 to 3, the inner cream has a shape that is enveloped by the beauty gel in the outer layer part, and oil soluble fragrance is added only to the cream part inside the bilayer gel cream. Thus, in the usage trials, it was confirmed that the bilayer gel cream before use was a formulation of which scent was hardly felt, but the fresh scent spread immediately after use through the usage trials. Further, the majority of the inner cream part was enveloped by a highly transparent gel even after use, and the freshness of the scent was maintained to the end. Furthermore, it was confirmed that the oily component of the bilayer gel cream is extremely less likely to directly come into contact with the external air, oxidation of the oily component contained in the inner cream is also suppressed, and oxidized odor of the oily component due to deterioration over time is not felt.

### (Functionality 7)

It was confirmed that the bilayer gel cream of Examples 1 to 20 also has an outward appearance with superb aesthetics in which a spherical cream part is completely surrounded by highly transparent beauty gel as shown in FIG. 2 to FIG. 43. It was also confirmed that the bilayer gel cream can be used without impairing the aesthetics thereof even during use.

In this manner, the present invention has been exemplified while using preferred embodiments of the present invention. However, the present invention should not be interpreted while being limited to the embodiments. It is understood that the scope of the present invention should be interpreted by the Claims. It is understood that those skilled in the art can implement an equivalent scope from the descriptions of the specific preferred embodiments of the invention based on the description of the present invention and common general knowledge.

### INDUSTRIAL APPLICABILITY

The present invention can provide a cosmetic material in which a gel-like, cream-like or semisolid formulation includes gel, or a cream-like or semisolid formulation in a spherical shape having a different composition and functionality. The cosmetic material of the present invention is a cosmetic material having functionalities of both two types of cosmetic materials in a single formulation as well as superb aesthetics.

### REFERENCE SIGNS LIST

1 Cosmetic product
2 Container
A Formulation in the outer layer
B Inner formulation

1.-13. (canceled)

14. A cosmetic product having a container, a transparent or semitransparent formulation A loaded into the container, and a formulation B embedded in the formulation A, wherein:
the formulation A is an aqueous solution comprising
(A) acrylic acid/alkylmethacrylic acid copolymer: 0.7 (% by weight),
(A) concentrated glycerin: 15 (% by weight),
(A) 1,3-butylene glycol: 5 (% by weight),
(A) methyl parahydroxybenzoate: 0.15 (% by weight),
(B) potassium hydroxide: 0.26 (% by weight); and
the formulation B is an aqueous solution comprising
(A) glyceryl tri-2-ethylhexanoate: 11 (% by weight),
(A) cetyl 2-ethylhexanoate: 5 (% by weight),
(A) methyl polysiloxane: 0.5 (% by weight),
(A) behenyl alcohol: 5 (% by weight),
(A) cetanol: 1 (% by weight),
(A) lipophilic glyceryl monostearate: 2.5 (% by weight),
(A) propyl parahydroxybenzoate: 0.2 (% by weight),
(A) natural vitamin E: 0.1 (% by weight),
(A) hydrogenated soybean phospholipid: 0.3 (% by weight),
(B) acrylic acid/alkylmethacrylic acid copolymer: 0.23 (% by weight),
(B) concentrated glycerin: 10 (% by weight),
(B) 1,3-butylene glycol: 5 (% by weight),
(B) maltitol laurate: 0.34 (% by weight),
(B) dehydrated ethanol: 0.07 (% by weight),
(B) N-stearoyl-L-sodium glutamate: 0.5 (% by weight),
(B) methyl parahydroxybenzoate: 0.2 (% by weight),
(C) potassium hydroxide: 0.081 (% by weight),
wherein the shape of the formulation B is a shape selected from the group consisting of a perfectly spherical shape, a spherical shape, an egg shape, a spheroid shape, a shape having an oval cross-section, a perfectly spherical shape with a protrusion on a surface, a spherical shape with a protrusion on a surface, an egg shape with a protrusion on a surface, a spheroid shape with a protrusion on a surface, and a shape having an oval cross-section with a protrusion on a surface, and
wherein the formulation B is added at 10 to 60% by weight of an entire formulation amount.

15. A cosmetic product having a container, a transparent or semitransparent formulation A loaded into the container, and a formulation B embedded in the formulation A, wherein:
the formulation A is an aqueous solution comprising
(A) acrylic acid/alkylmethacrylic acid copolymer: 0.7 (% by weight),
(A) concentrated glycerin: 15 (% by weight),
(A) 1,3-butylene glycol: 5 (% by weight),
(A) methyl parahydroxybenzoate: 0.15 (% by weight),
(A) phenoxyethanol: 0.3 (% by weight),
(B) potassium hydroxide: 0.26 (% by weight); and
the formulation B is an aqueous solution comprising
(A) glyceryl tri-2-ethylhexanoate: 11 (% by weight),
(A) cetyl 2-ethylhexanoate: 5 (% by weight),
(A) methyl polysiloxane: 0.5 (% by weight),
(A) behenyl alcohol: 5 (% by weight),
(A) cetanol: 1 (% by weight),
(A) lipophilic glyceryl monostearate: 2.5 (% by weight),
(A) propyl parahydroxybenzoate: 0.2 (% by weight),
(A) natural vitamin E: 0.1 (% by weight),
(A) hydrogenated soybean phospholipid: 0.1 (% by weight),
(B) acrylic acid/alkylmethacrylic acid copolymer: 0.23 (% by weight),
(B) xanthan gum: 0.1 (% by weight),
(B) concentrated glycerin: 10 (% by weight),
(B) 1,3-butylene glycol: 5 (% by weight),
(B) phenoxyethanol: 0.3 (% by weight),
(B) N-stearoyl-L-sodium glutamate: 0.8 (% by weight),
(B) propyl parahydroxybenzoate: 0.2 (% by weight),
(C) potassium hydroxide: 0.081 (% by weight),
wherein the shape of the formulation B is a shape selected from the group consisting of a perfectly spherical shape, a spherical shape, an egg shape, a spheroid shape, a shape having an oval cross-section, a perfectly spherical shape with a protrusion on a surface, a spherical shape with a protrusion on a surface, an egg shape with a protrusion on a surface, a spheroid shape with a protrusion on a surface, and a shape having an oval cross-section with a protrusion on a surface, and
wherein the formulation B is added at 10 to 60% by weight of an entire formulation amount.

16. A cosmetic product having a container, a transparent or semitransparent formulation A loaded into the container, and a formulation B embedded in the formulation A, wherein:
the formulation A is an aqueous solution comprising
(A) acrylic acid/alkylmethacrylic acid copolymer: 0.7 (% by weight),
(A) concentrated glycerin: 15 (% by weight),
(A) 1,3-butylene glycol: 1 (% by weight),
(A) 1,2-pentanediol: 4 (% by weight),
(B) potassium hydroxide: 0.26 (% by weight); and
the formulation B is an aqueous solution comprising
(A) glyceryl tri-2-ethylhexanoate: 11 (% by weight),
(A) cetyl 2-ethylhexanoate: 5 (% by weight),
(A) methyl polysiloxane: 0.5 (% by weight),
(A) behenyl alcohol: 5 (% by weight),
(A) cetanol: 1 (% by weight),
(A) lipophilic glyceryl monostearate: 2.5 (% by weight),
(A) natural vitamin E: 0.1 (% by weight),
(A) hydrogenated soybean phospholipid: 0.1 (% by weight),
(B) acrylic acid/alkylmethacrylic acid copolymer: 0.23 (% by weight),
(B) xanthan gum: 0.1 (% by weight),
(B) concentrated glycerin: 10 (% by weight),
(B) 1,3-butylene glycol: 1 (% by weight),
(B) 1,2-pentanediol: 4 (% by weight),
(B) maltitol laurate: 0.34 (% by weight),
(B) dehydrated ethanol: 0.07 (% by weight),
(B) N-stearoyl-L-sodium glutamate: 0.5 (% by weight),
(C) potassium hydroxide: 0.081 (% by weight),
wherein the shape of the formulation B is a shape selected from the group consisting of a perfectly spherical shape, a spherical shape, an egg shape, a spheroid shape, a shape having an oval cross-section, a perfectly spherical shape with a protrusion on a surface, a spherical shape with a protrusion on a surface, an egg shape with a protrusion on a surface, a spheroid shape with a protrusion on a surface, and a shape having an oval cross-section with a protrusion on a surface, and
wherein the formulation B is added at 10 to 60% by weight of an entire formulation amount.

17. The cosmetic product of claim 14, wherein the formulation B is embedded while floating in the formulation A.

18. The cosmetic product of claim 14, wherein the formulation B is added at 20 to 40% by weight of an entire formulation amount.

19. The cosmetic product of claim 14, wherein a maximum load indicating a formulation viscosity of the formulation A and the formulation B measured under the following conditions is 0.3 N to 2.0 N:
a maximum value of load applied per cross-sectional area of a rod-like probe with a diameter of 20 mm when the rod-like probe is inserted 25 mm at a rate of 60 mm/min as measured with Sun Scientific Co., Ltd.'s SUN RHEO METER (COMPAC 100-II).

20. The cosmetic product of claim 15, wherein the formulation B is embedded while floating in the formulation A.

21. The cosmetic product of claim 15, wherein the formulation B is added at 20 to 40% by weight of an entire formulation amount.

22. The cosmetic product of claim 15, wherein a maximum load indicating a formulation viscosity of the formulation A and the formulation B measured under the following conditions is 0.3 N to 2.0 N:
a maximum value of load applied per cross-sectional area of a rod-like probe with a diameter of 20 mm when the rod-like probe is inserted 25 mm at a rate of 60 mm/min as measured with Sun Scientific Co., Ltd.'s SUN RHEO METER (COMPAC 100-II).

23. The cosmetic product of claim 16, wherein the formulation B is embedded while floating in the formulation A.

24. The cosmetic product of claim 16, wherein the formulation B is added at 20 to 40% by weight of an entire formulation amount.

25. The cosmetic product of claim 16, wherein a maximum load indicating a formulation viscosity of the formulation A and the formulation B measured under the following conditions is 0.3 N to 2.0 N:
a maximum value of load applied per cross-sectional area of a rod-like probe with
a diameter of 20 mm when the rod-like probe is inserted 25 mm at a rate of 60 mm/min as measured with Sun Scientific Co., Ltd.'s SUN RHEO METER (COMPAC 100-II).
